(19) United States
(12) Patent Application Publication
(10) Pub. No.: US 2020/0229407 A1
(43) Pub. Date: Jul. 23, 2020

(54) PRODUCTS AND METHODS ASSOCIATED WITH MULTIPLE SCLEROSIS AS A TRANSMISSIBLE PROTEIN MISFOLDING DISORDER

(71) Applicant: Amira Medical Technologies Inc., Calgary (CA)

(72) Inventors: Peter Stys, Calgary (CA); Gerald W. Zamponi, Calgary (CA); Shigeki Tsutsui, Calgary (CA)

(21) Appl. No.: 16/754,954

(22) PCT Filed: Oct. 12, 2018
(86) PCT No.: PCT/CA2018/051287

§ 371 Date: Apr. 9, 2020

Related U.S. Application Data
(60) Provisional application No. 62/571,987, filed on Oct. 13, 2017.
(60) Provisional application No. 62/572,104, filed on Oct. 13, 2017.

(51) Int. Cl. A01K 67/027; G01N 33/68; A61K 31/454; A61K 31/473; A61K 31/65; A61K 31/737; C07K 14/47; C12Q 1/37

(52) U.S. Cl. CPC A01K 67/0275; G01N 33/6896; A61K 31/454; A61K 31/473; A61K 31/65; A61K 31/737; G01N 2800/285; C12Q 1/37; A01K 2207/10; A01K 2217/05; A01K 2227/105; A01K 2267/0343; G01N 2500/10; C07K 14/47

(57) ABSTRACT

Described are methods and products related to the identification of multiple sclerosis (MS) as a transmissible protein misfolding disorder. Data is presented to support the position that the transmissible protein is an abnormal prion protein conformer (PrP^{MS}). Methods are described for identifying a subject having, or at risk of developing, multiple sclerosis (MS) based on determining the presence or absence of PrP^{MS} in a sample from the subject. The presence of the abnormal prion protein conformer in the sample is indicative of the subject having MS or an increased risk of developing MS. Also described are therapeutics methods for the treatment of MS as well as cell cultures, non-human animal models and biological samples thereof useful for the study of MS.

### RELATED APPLICATIONS

This application claims the benefit of priority to U.S. Provisional Patent Application No. 62/571,987 filed on Oct. 13, 2017 and to U.S. Provisional Patent Application No. 62/572,104 filed on Oct. 13, 2017, the contents of which are hereby incorporated by reference in their entirety

### FIELD OF THE INVENTION

The present invention relates to multiple sclerosis and more specifically to methods, uses and products associated with multiple sclerosis as a transmissible protein misfolding disorder.

### BACKGROUND OF THE INVENTION

Multiple Sclerosis (MS) is the commonest cause of progressive neurological disability in young adults with a world-wide prevalence of over 2 million individuals [1]. Despite immense research effort, the etiology remains unknown. Traditionally considered to be an autoimmune inflammatory demyelinating disorder of the CNS, MS histopathology includes prominent T-cell inflammation, macrophage infiltration, and demyelination as a universal finding [2]. Neuro-axonal degeneration is also widespread [3,4]. At onset, MS exhibits a fluctuating course, in both space and time, with most patients presenting with relapsing and remitting attacks of inflammatory demyelination. This initial phase is followed by chronic progression in most patients, with quiescence of inflammation [5]. Our understanding of the immunobiology of MS is mature [6,7]. Large genome-wide association studies point to a role for many immune-related genes [8], and several environmental factors, presumably acting via immune modulation, are associated with MS risk [9,10]. Taken together, a large body of data points to an autoimmune component, which paved the way for development of drugs that are highly effective at reducing inflammation and relapses. Unfortunately, these therapies are largely ineffective in preventing irreversible disability in the later progressive phase [11] where inflammation and autoimmunity are less prominent. A major challenge of MS research is to better understand progressive disease in order to extend diagnostic and therapeutic options.

The cellular prion protein (PrPc) is a normal protein highly expressed in the mammalian CNS, found at synapses, on neurons, axons, oligodendrocytes and myelin [29-33]. Mature PrPc is a glycoprotein that is anchored extracellularly, containing several Cu binding octarepeats [34,35]. Despite its abundance, its normal role is poorly understood, but may include Cu homeostasis and protection against injury from a variety of insults [36-40]. PrPc modulates several neurotransmitter receptors [41], notably N-methyl-D-aspartate receptors NMDARs [36,42]. Misfolding of PrPc into a protease-resistant “scrapie” conformation (PrP^{Sc}), occurring sporadically or due to mutations of the PRNP gene, causes a number of neurodegenerative diseases targeting all levels of the nervous system, including brain, spinal cord, PNS and even enteric [43-46]. These transmissible spongiform encephalopathies (TSEs) are but one group of a growing collection of “protein aggregation diseases”, which also includes many common degenerations such as Alzheimer's, Parkinson's, tauopathies and ALS [47,48].

There is a need for novel approaches for the study, diagnosis and treatment of MS.

### SUMMARY OF THE INVENTION

In one aspect of the invention, multiple sclerosis has been identified as a transmissible protein misfolding disorder. In one embodiment, prion aggregates have been shown to be associated with multiple sclerosis (MS). Remarkably, transgenic mice that overexpress human prion protein (PrP) injected with brain homogenate from a secondary PPMS patient developed brain atrophy and ventriculomegaly (commonly seen in MS patients) as well as plaque-like PrP deposits not seen in controls. Passaging and transmission of an MS-like pathology was also observed by injecting naïve transgenic mice with brain homogenate from infected mice. Mice injected with MS brain homogenate exhibited abnormal behavior including deficits in spatial learning and memory and decreased exploratory behavior/more anxiety. Transmission of an MS-like pathology was also observed by injecting naïve transgenic mice with MS spinal cord homogenate.

Accordingly, in one aspect there is provided a method of identifying a subject having, or at risk of developing, multiple sclerosis (MS). In one embodiment, the method comprises determining the presence or absence of an abnormal prion protein conformer (PrP^{MS}) in a sample from the subject. In one embodiment, the presence of the abnormal prion protein conformer in the sample is indicative of the subject having MS or an increased risk of developing MS.

The abnormal prion protein conformer (PrP^{MS}) described herein can readily be distinguished from prion protein that is non-pathogenic or not associated with MS or an increased risk of developing MS. For example, in one embodiment, the abnormal prion protein conformer forms deposits of β-sheet rich amyloid. In one embodiment, samples comprising the abnormal prion protein conformer (PrP^{MS}) cause transmission of MS pathology to a test subject when administered to a test subject. The PrP^{MS} conformer or aggregates thereof can also be detected in vitro. For example, in one embodiment, contacting a culture of central nervous system (CNS) tissue with PrP^{MS} causes the production of amyloid deposits in the cell culture. In one embodiment, the CNS tissue is brain tissue. In one embodiment, Photo-Induced Cross-Linking of Unmodified Proteins (PICUP) is used to stabilize PrP^{MS} aggregates for biochemical detection. In one embodiment, protein misfolding cyclic amplification (PMCA) is used to amplify the amount of PrP^{MS} in a sample. Optionally, techniques such as PICUP and/or PMCA may be used in combination with techniques known in the art for detecting prion aggregates such as Western Blotting, prion specific binding reagents such as antibodies, PTA precipitation, and/or spectral analysis.

In one embodiment, the abnormal prion protein conformer may be detected using spectral analysis. For example, in one embodiment a conformationally-sensitive probe that binds to protein aggregates is used wherein binding of the probe to PrP^{MS} aggregates causes a change in the spectral properties of the probe. In one embodiment, the PrP^{MS} conformer may be detected using real-time quaking-induced conversion (RT-QuIC). In one embodiment, the PrP^{MS} conformer may be detected using antibodies and/or biochemical techniques such as protease digestion and fragment analysis.

In one embodiment, the method further comprises treating and/or monitoring a subject identified as having MS or an increased risk of developing MS. In one embodiment, the method comprises monitoring the presence, absence and/or level of the PrP^{MS} conformer in the subject over time.

In one embodiment, there is provided a system for identifying a subject having, or at risk of developing, multiple sclerosis (MS). In one embodiment, the system comprises a processor configured to compare spectra of a sample from the subject in contact with a conformationally sensitive probe that binds to PrP and one or more reference spectra.

In one aspect, there is provided a cell and/or cell culture that has been isolated and/or modified to contain a transmissible protein from a subject with MS that results in MS pathology. In one embodiment, there is provided a cell and/or cell culture that has been isolated and/or modified to contain PrP^{MS}. In one embodiment, cells, cell cultures and/or tissue cultures that contain PrP^{MS} are useful as a model for the study of MS. In one embodiment, PrP^{MS} conformers may be used to infect and/or transmit PrP^{MS} conformations to naïve cells and/or cell cultures to promote the formation of PrP^{MS} conformers and/or aggregates from endogenous PrP.

In one embodiment, there is provided a cell culture produced by contacting one or more cells with a composition comprising a homogenate from a subject with MS, optionally a brain homogenate or a spinal cord homogenate, and culturing the cells. In one embodiment, there is provided a cell culture produced by contacting one or more cells with a composition comprising PrP^{MS} and culturing the cells. In one embodiment, the composition comprising PrP^{MS} is a brain homogenate from a subject with MS. In one embodiment, the composition comprising PrP^{MS} is derived from a PrP^{MS} cell culture as described herein. In one embodiment the cell culture comprises, consists essentially of, or consists of cells of a single type. In another embodiment, the cell culture comprises, consists essentially of, or consists of cells of a plurality of different types.

In one embodiment the cells are brain cells such as neurons, astrocytes, oligodendrocytes, oligodendrocyte precursor cells and/or microglia. In one embodiment, the cells are immune cells such as B cells and/or Follicular Dendritic cells, optionally alone or in combination with brain cells. In one embodiment, the cells are a culture of central nervous system (CNS) tissue, optionally a slice culture of CNS tissue. In one embodiment, the tissue or slice culture comprises cortical tissue, corpus callosum tissue, cerebellar tissue, ex vivo optic nerve tissue and/or spinal dorsal column/dorsal root ganglion (DRG) tissue. In one embodiment, the cells are recombinant and/or transgenic cells. In one embodiment, the cells express or overexpress prion protein, optionally human prion protein. In one embodiment, the cells are immortalized cells, optionally immortalized cells that have been genetically engineered to overexpress PrP.

In one aspect, the cells and/or cell cultures described herein are useful for screening compounds for identifying diagnostic agents for the detection of MS and/or therapeutic agents for the treatment of MS. In one embodiment, there is provided a method comprising contacting a PrP^{MS} cell culture as described herein with a test compound and detecting a biological effect of the test compound on the PrP^{MS} cell culture. In one embodiment, the biological effect is a reduction in the presence of PrP^{MS} and/or amyloid-like deposits in the PrP^{MS} cell culture relative to a control. In one embodiment, the biological effect is a modification of cytotoxicity, cytodegeneration or cell viability assays in the presence of PrP^{MS} and/or amyloid-like deposits in the PrP^{MS} cell culture relative to a control. In one embodiment, the method comprises identifying a test compound that has a biological effect on the PrP^{MS}cell culture as a candidate therapeutic agent for the treatment of MS.

In another aspect, there is provided a non-human animal model of multiple sclerosis (MS). In one embodiment, the animal model is produced by administering a composition comprising a brain homogenate or spinal cord homogenate from a subject with MS to a non-human animal. In one embodiment the animal model is produced by administering a composition comprising an abnormal prion protein conformer (PrP^{MS}) to a non-human animal. In one embodiment, the non-human organism is a mammal, optionally a rodent such as a mouse or a bank vole. In one embodiment, the non-human animal is a transgenic animal, optionally a transgenic organism that overexpresses prion protein such as human prion protein or bank vole prion protein. Also provided are cell cultures obtained from the non-human animal models described herein.

The non-human animal models of MS described herein are useful for screening compounds for the identification of diagnostic agents for the detection of MS and/or therapeutic agents for the treatment of MS.

Accordingly, in one embodiment, there is provided a method comprising administering a test compound to a non-human model organism of MS as described herein and detecting a biological effect of the test compound on the non-human animal model of MS.

In one embodiment, the biological effect is an increase or decrease in one or more signs or symptoms of MS in the model animal. For example, in one embodiment the signs or symptoms of multiple sclerosis include one or more of amyloid plaques, brain atrophy, demyelination, microgliosis, proliferation of oligodendrocyte precursors, ventriculomegaly and abnormal animal behavior. In one embodiment, the biological effect is a reduction in the presence of PrP^{MS} conformer and/or amyloid-like deposits in the non-human model animal relative to a control animal. In one embodiment, the biological effect is demyelination and/or degeneration, optionally measured through Magnetic Resonance Imaging (MRI), Computerized Tomography (CT) and/or Micro/Nano Computed Tomography (micro-CT and/or nano-CT).

Given that PrP^{MS} misfolded prion conformers are associated with the pathology of multiple sclerosis, agents that reduce or inhibit the formation of PrP^{MS} conformers may be useful for the treatment or prevention of MS.

Accordingly, in one aspect there is provided a method for the treatment or prevention of multiple sclerosis. In one embodiment, the method comprises administering to the subject an agent that reduces and/or inhibits the formation of PrP^{MS} conformers. Also provided is the use of an agent that reduces and/or inhibits the formation of PrP^{MS} conformers in a subject in need thereof. In one embodiment, the agent that reduces and/or inhibits the formation of abnormal prion protein conformer PrP^{MS} is listed in Table 1. In one embodiment, the agent listed in Table 1 has been chemically modified and/or formulated to cross the blood brain barrier.

In another aspect, there is provided a method for reducing and/or inhibiting the formation of PrP^{MS} conformers in central nervous system (CNS) cells or tissue. In one embodiment, the method comprises contacting CNS cells or tissue with an agent listed in Table 1. In one embodiment, the CNS cells or tissue are in vivo or in vitro. In one embodiment, the CNS cells are brain cells and/or immune cells. Also provided is the use of an agent listed in Table 1 for reducing and/or inhibiting the formation of PrP^{MS} conformers in vivo or in vitro.

Other features and advantages of the present invention will become apparent from the following detailed description. It should be understood, however, that the detailed description and the specific examples while indicating preferred embodiments of the invention are given by way of illustration only, since various changes and modifications within the spirit and scope of the invention will become apparent to those skilled in the art from this detailed description.

### BRIEF DESCRIPTION OF THE DRAWINGS

Embodiments of the invention will now be described in relation to the drawings in which:

FIG. 1 shows amyloid microspectroscopy and associated analysis. The conformationally-sensitive fluorescent amyloid probe pFTAA was used to stain human control and MS brain. Spectral micrographs were acquired and displayed in monochrome with their spectral differences emphasized (dark gray: normal brain parenchyma, light gray: mature amyloid). A,B: Control brain has only non-specific RBC autofluorescence (denoted by *); neurons are plentiful but barely visible (A). Background is dark gray indicating little amyloid deposition. C: In contrast, PPMS gray matter contains many light gray neurons suggesting early amyloid accumulation in their somata. D: PPMS white matter has widespread fibrillar amyloid deposition with a perivascular (V) predilection (arrows). PPMS background is shifted to light gray, suggesting widespread parenchymal amyloid deposition. E: Quantitative “amyloid index”: “Gray/white matter” includes the entire image (except for autofluorescent RBCs), and “neuropil” is background parenchyma excluding all cell bodies, and the prominent perivascular deposit in D reflecting the “normal appearing” parenchyma. F: Basis spectra from normal and MS brain. These results indicate that PPMS brain has widespread deposition of abnormally aggregated toxic amyloid, presumably PrP. These background abnormalities are generally invisible to conventional histopathological examination.

FIG. 2 shows mouse brain 9.4T MRI images and associated analysis. A-D: quantitative T2 MRI of four tg650 mouse brains 10 months after inoculation with human control (A,B) or MS brain homogenate (C,D). Marked cortical atrophy (arrows) and ventriculomegaly were only seen in the MS-injected animals. E,F: T2 decay times from various regions in E. The more unrestricted water a tissue contains, the longer its T2 decay; this constitutes a sensitive measure of tissue pathology/microedema. Voxels are processed and assigned an intensity (y-axis) and an index (x-axis) mirroring the decay time. A 2D histogram shows the various populations: note 2 distinct clusters at the lower left that are distinguishable (muscle, brain). This analysis can be restricted to brain regions of interest. Voxels from normal appearing cortex (H) and corpus callosum (I) exhibit subtle shifts to higher intensity/longer decays. Placing regions of interest (rectangles) on such scatterplots allows statistical comparisons between groups. An overall pathology score is calculated as the product of intensity x index for each voxel (J). This allows for the detection of subtle regional brain pathology non-invasively, in the absence of anatomical abnormalities, in the same animal followed serially over time.

FIG. 3 shows inoculated mouse brain histology. tg650 mice injected i.c. with either control (left panels) of MS (right panels) brain homogenate and allowed to incubate for ≈1 year. Samples were treated with formic acid to remove normal PrPc from tissue, leaving pathological resistant PRP. Immunohistochemistry for PrP (dark gray signal) after treatment shows virtually complete absence in control, but extensive abnormal deposits in MS-infected mouse brain.

FIG. 4 shows that spin column fractionation of MS-inoculated tg650 brain exhibited a shift to higher molecular weight complexes indicating the presence of an abnormally-aggregated PrP (PrP^{MS}).

FIG. 5 shows the protease resistance of PrP from tg650 mice injected with human brain homogenates from healthy controls (left) or MS patients (right). Brain homogenates were treated with increasing concentrations of Pronase E. Controls show near complete digestion of PRP by 30 μg/ml treatment. MS samples indicate the presence of lower molecular weight products that are resistant to digestion.

FIG. 6 shows results of precipitation of abnormally aggregated PrP from human brain injected tg650 mice. A: Brain homogenates from negative control (C57 WT) or MS-infected tg650 mice were processed with PTA (phosphotungstic acid) which precipitates only abnormally aggregated PrP. Aggregated PrP was identified only in the tg650 sample. B: A novel assay that couples PTA precipitation with fluorescent amyloid probes in a 96-well plate setup. MS samples (light gray) were strongly positive vs. human and mouse controls.

FIG. 7 shows that intraperitoneal (i.p.) injection is sufficient to produce MS related pathology in mouse brain. Human control brain (A) or MS brain were injected i.p. into tg650 mice. MS-brain injected mice show multifocal T2 hyperintense legions (arrows), many of which involve the corpus callosum. Circles at top left are CuSO₄ phantoms used to calibrate intensity and decay times for highly quantitative analysis.

FIG. 8 shows that MS brain injection induces cerebellar atrophy. Mouse brain MRI of control (A) and MS brain-injected tg650 mice (B) shows fourth ventricular dilatation in the latter reflecting atrophy of posterior brain structures such as cerebellum, confirmed by H&E staining: marked atrophy of cerebellar folia (denoted by *) with vacuolar/spongiform change in the deeper cerebellar white matter was seen in MS-injected mouse cerebellum (D), not in control (C). Importantly, no appreciable lymphocytic infiltration was seen.

FIG. 9 shows a spectral micrograph and analysis of isolated leukocytes from MS patients stained with pFTAA. Spectral micrograph is displayed with emphasis on red end of spectra. Characteristic inclusions that are likely aggregates of misfolded protein. 9B shows basis spectra used for the analysis with left-shifted spectrum from control leukocytes (dark gray) vs. red granules from MS (light gray). 9C shows the results from using a non-linear algorithm to scan spectral images of stained leukocyte smears in an unbiased manner to calculate an “MS disease score”. All controls were below an arbitrary threshold for this dataset, including two Alzheimer's cases that are positive using different probes and basis spectra, underscoring the specificity of this technique. Five of 6 MS cases were unequivocally positive.

FIG. 10 shows that MS brain injection induced demyelination of the corpus callosum. Demyelinating pathology in controls (A) vs. MS (B) -injected Tg650 mouse brain (1st passage). Corpus callosum often exhibited evidence of demyelination in MS brain-injected mice, but never in control (H&E-LFB, QD-9 and SMI-94), paralleling the 9.4T MRI quantitative T2 imaging shown in E. C) and D): Quantitative analyses of degenerated myelin (QD9/MBP) and loss of MBP (SMI-94) immunohistochemical pathology, which are characteristic of human MS.

FIG. 11 shows MS brain-induced human MS-like neuropathology. Representative immunofluorescence micrographs of human MS-like neuropathology in tg650 mouse. A) Microglial activation in MS-injected tg650 mouse brain. Iba-1-positive cells were prominent in periventricular white matter after MS brain homogenate inoculation. B) Anti-non-phosphorylated neurofilament antibody (SMI32) was used as a marker of axonal injury. Control injected brain had strong myelin signal and minimal nonphosphorylated neurofilament. In contrast, MS-inoculated mice exhibited significant myelin injury reflected by loss of MBP, in both gray and white matter, together with a marked increase in injured axons. C: Quantitative analyses of microglial (Iba1) and axonal (SMI-32) immunohistochemical pathology. These patterns are also characteristic of human MS.

FIG. 12 shows that MS brain-injected tg650 mice exhibited abnormal behavior. MS brain-injected animals exhibited deficits in spatial learning and memory on water maze testing (A) and decreased exploratory behavior/more anxiety (B).

FIG. 13 shows MS brain injection induced abnormal PrP accumulation. Immunohistochemical staining of prion protein with 3F4 antibody in tg650 mouse brain after prior inoculation with either human control or MS brain (1^{st} passage). Mouse brain sections were treated with formic acid (FA) to abolish background PrPc signal. Representative images showing no FA-resistant PrP in tg650 brain originally inoculated with control human brain. In contrast, tg650 mouse brain that was initially injected with human MS brain homogenate showed FA resistant deposits highly reminiscent of variably proteinase-resistant prionopathy (VPSPr). Such “PrP^{MS}” deposits were found in extracellular brain parenchyma and also in the perivascular spaces. Abnormal PrP^{MS} deposits were also observed after multiple passaging.

FIG. 14 shows 9.4 T quantitative T2 MRI analysis. Pathology scores (based on exponential decay analysis as in Kidd et al. [119]) showed significant abnormalities after i.c. injection of MS brain (1st passage, ≈1yr incubation, A) and also with 2nd passage of MS-infected mouse brain (≈6mo incubation; B) (each circle is a different injected mouse). Controls (including Alzheimer's & Lewy body disease, dark gray symbols, and importantly, pathological 1st passage brain homogenates immunodepleted of PrP, light gray symbols) did not induce pathology. Interestingly thalamus was very susceptible, paralleling observations in human MS.

FIG. 15 shows spinal cord pathology after intraperitoneal injection of MS homogenate. Transmission of pathology to spinal cord revealed by FA-resistant 3F4 immunohistochemistry of lumbar cord after ≈8 months incubation. tg650 mice injected either with non-MS spinal cord homogenate i.p. (A) or with MS brain homogenate but i.c. (B), showed no obvious pathology. In contrast, mice injected with MS spinal cord homogenate i.p. exhibited pronounced FA-resistant PrP deposits mainly in the superficial layers of the dorsal horns (C, arrows) where sensory afferents terminate. G-I: Significant myelin loss and synaptic damage were observed in MS-injected lumbar cord, whereas control-injected showed no pathology (D-F). J-L: Quantitative analyses of axonal (SMI-32) and microglial (Iba1, not significant) immunohistochemical pathology in the superficial layer of dorsal horns. This provides compelling evidence of a disease-associated factor in MS homogenate that is able to invade the CNS after i.p. inoculation, in a region-specific fashion, and in a manner similar to scrapie prions. MS-injected mice also exhibited significant pain hypersensitivity (data not shown). Interestingly, other brain homogenates induced strong 3F4 immunoreactivity mainly in spinal white matter, suggesting possible tissue/strain-specific tropism (M and N). N 5 mice for each group.

### DETAILED DESCRIPTION OF THE INVENTION

The inventors have surprisingly determined that subjects with multiple sclerosis (MS) exhibit abnormal deposits of prion protein (PrP) and that MS pathology is associated with a progressive transmissible prion protein folding disorder.

Without being limited by theory, it is believed that MS is caused by misfolding of normal prion protein (PrP^{C}) into an aggregation-prone conformer, PrP^{MS}, which accumulates in the brain of MS patients and causes dysregulation of myelinic GluRs and non-immune demyelination. The accumulation of pathological prion may induce chronic dysregulation of the axo-myelinic synapse culminating in progressive disease. Previously it was not known that abnormal PrP could be detected in subjects with MS nor has there been any biological data demonstrating disease transmission consistent with MS being a prionopathy. In contrast, a commonly accepted view is that MS is a primary autoimmune disease associated with genetic and/or environmental risk factors.

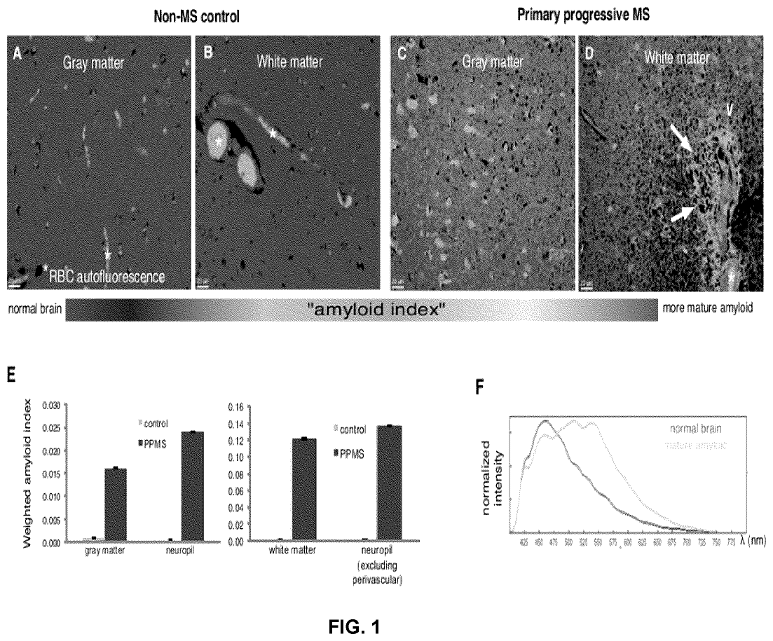

As set out in Example 1 and FIG. 1, microspectroscopy using conformationally-sensitive fluorescent probes that bind to misfolded amyloid plaques detected the presence of amyloid deposits in human MS brain samples not seen in controls.

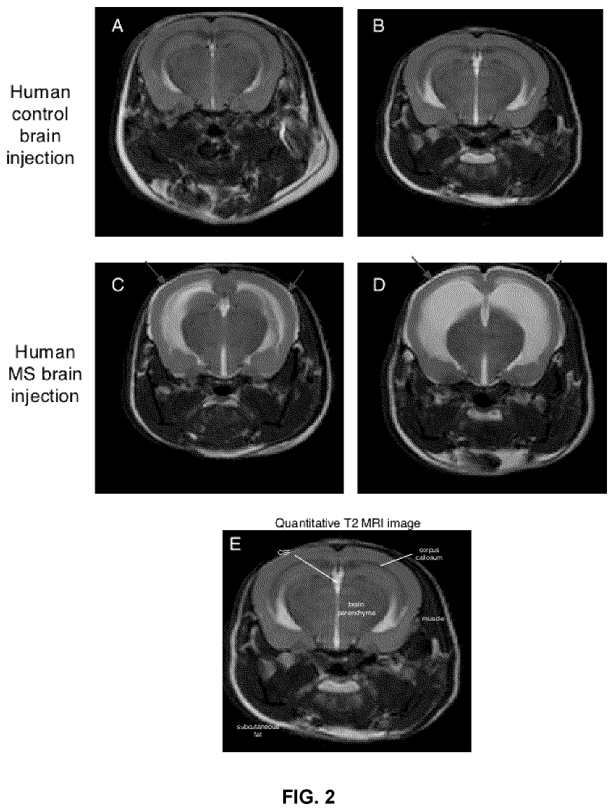

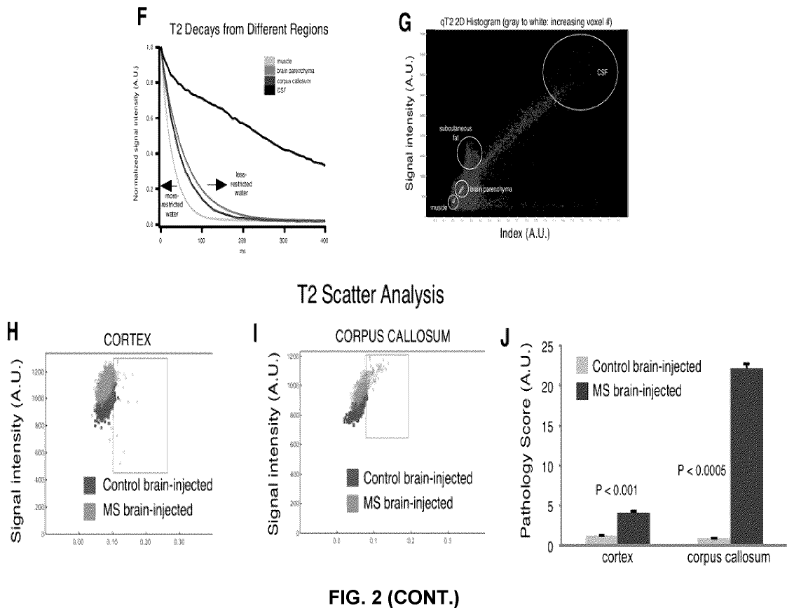

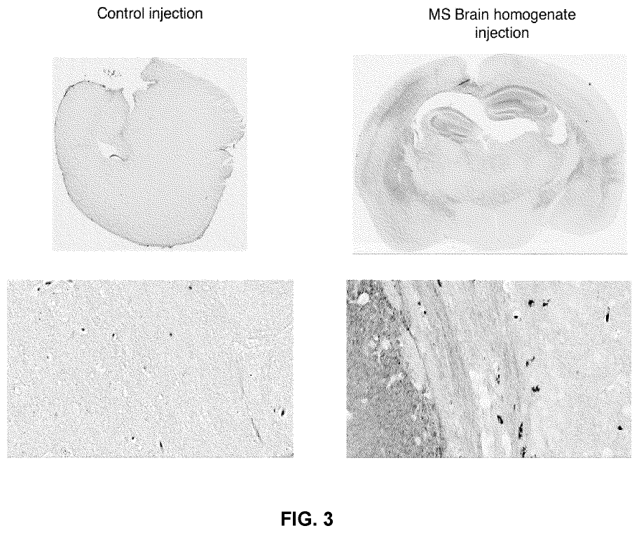

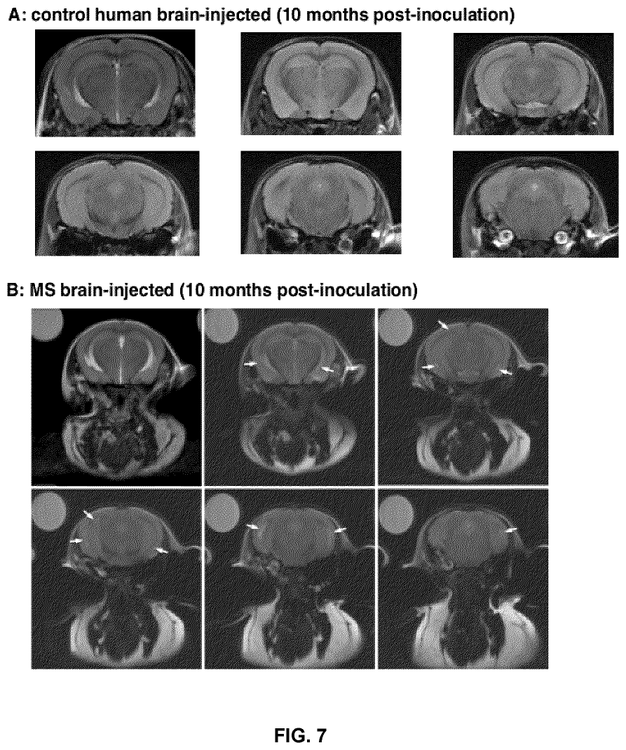

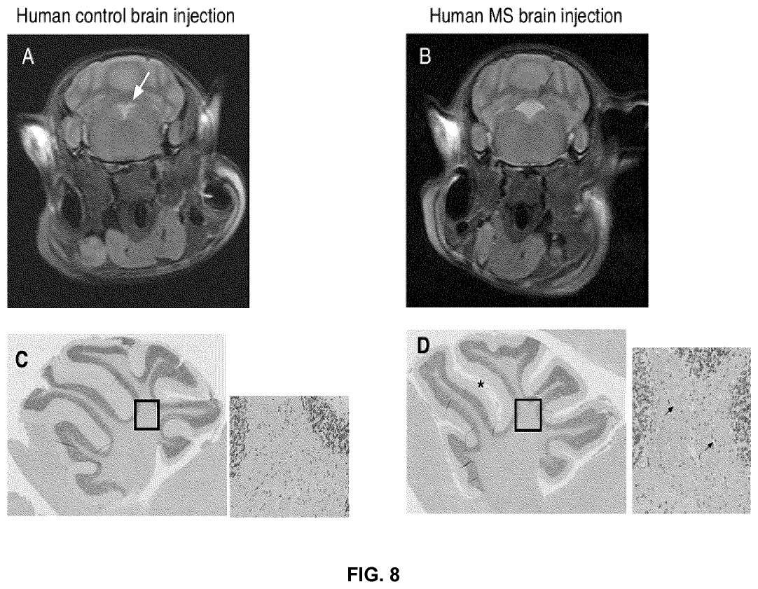

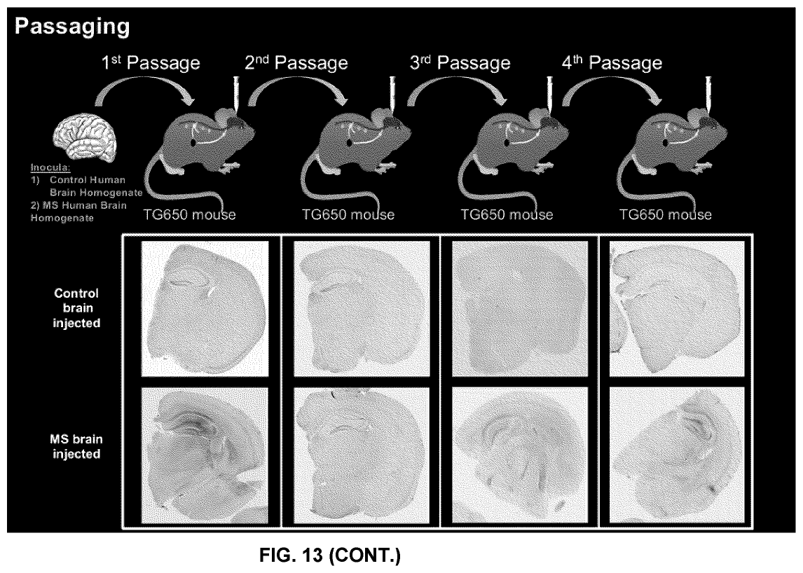

As set out in Example 2 and FIGS. 2-8 experiments using transgenic tg650 mice that overexpress human prion protein injected intracerebrally with a brain homogenate from a subject with secondary progressive MS demonstrated the transmission of MS-like pathology and the deposition of amyloid plaques. In addition, biochemical characterization of prion protein from MS-inoculated tg650 mouse brain suggests the presence of a distinct abnormally-aggregated conformer of PrP, identified herein as PrP^{MS}. Remarkably, abnormal PrP^{MS} deposits were observed after multiple passages in mice (FIG. 13).

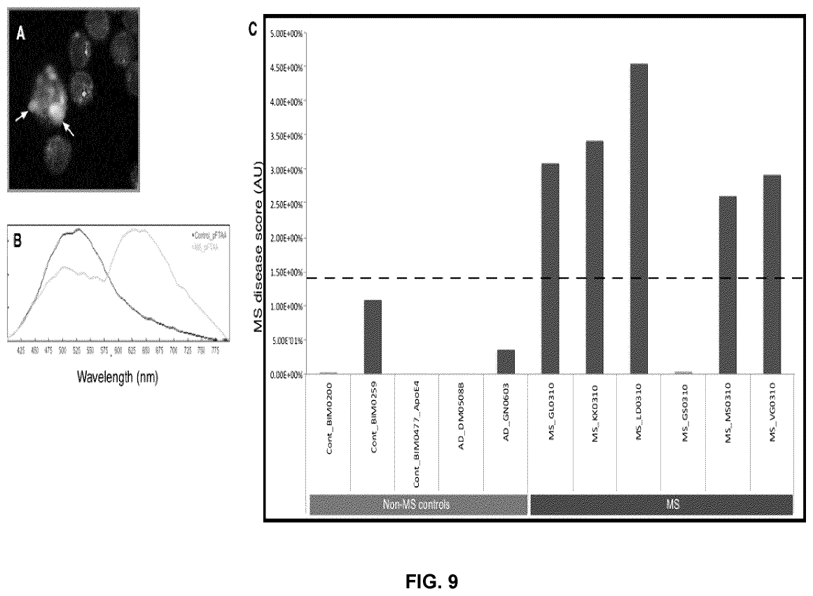

Furthermore, as shown in FIG. 9 spectral micrographs of leukocytes isolated from subjects with MS exhibited distinctive spectral signatures indicative of the presence of abnormally folded protein conformers.

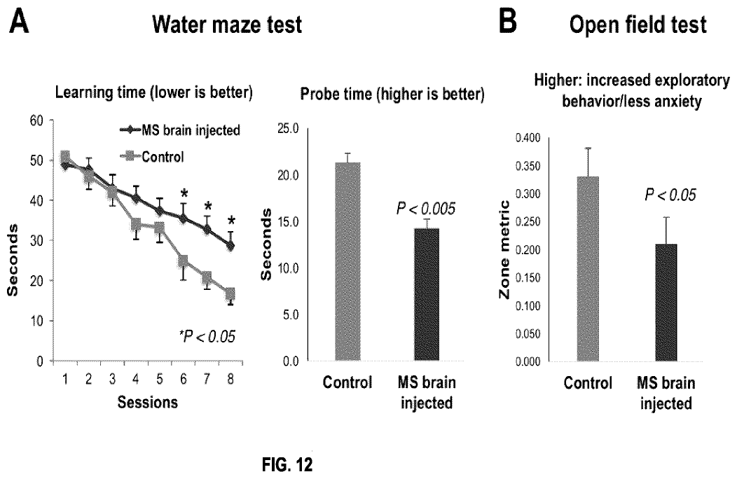

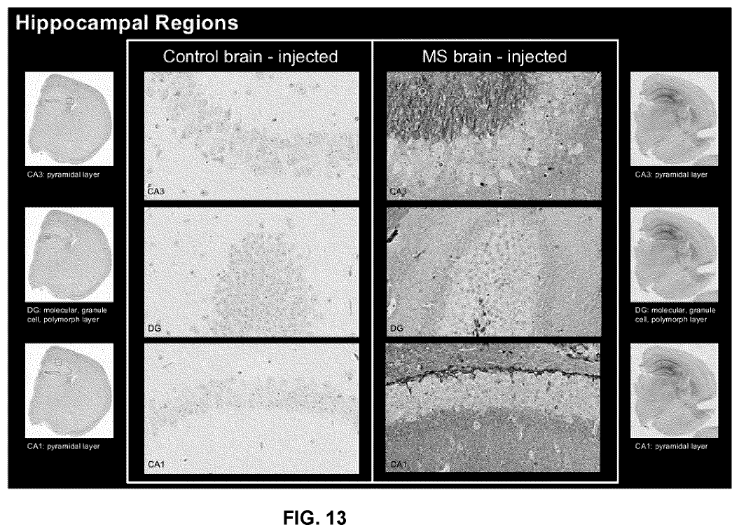

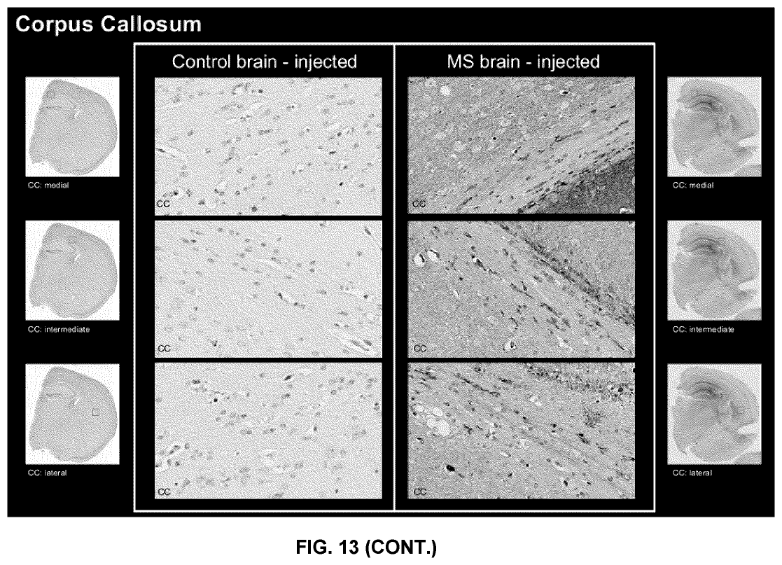

As set out in Example 6 and FIG. 12, MS brain-injected tg650 mice also exhibited abnormal behaviors including deficits in spatial learning and memory as well as decreased exploratory behavior and more anxiety.

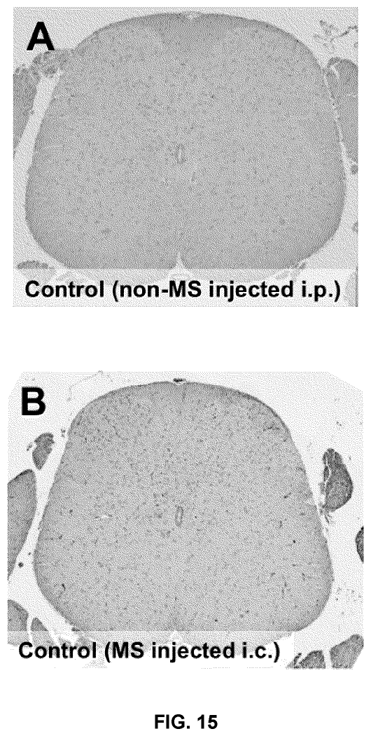

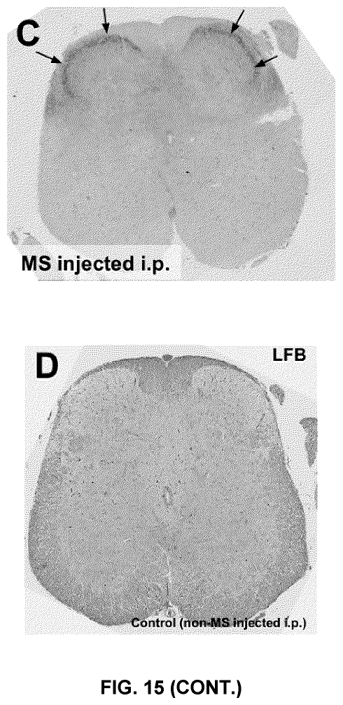

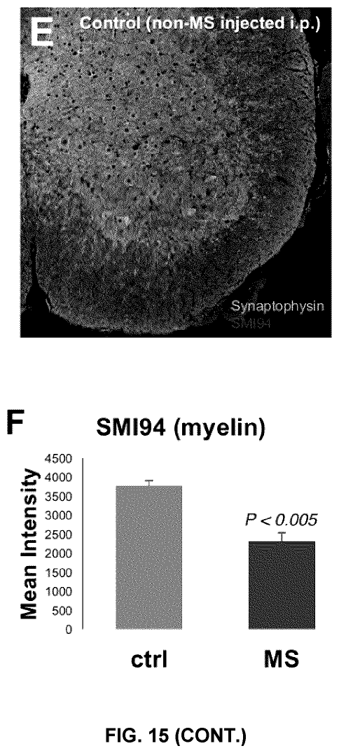

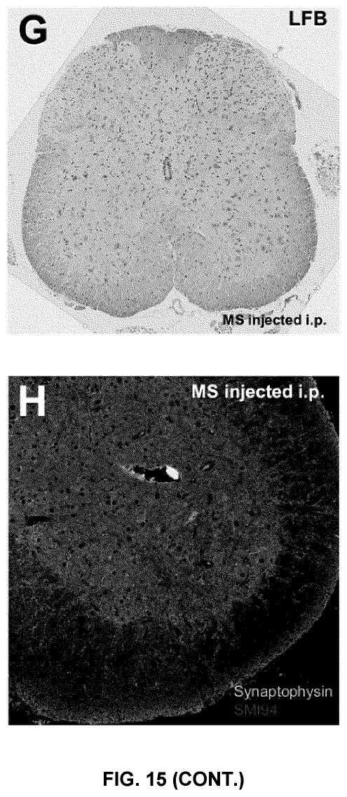

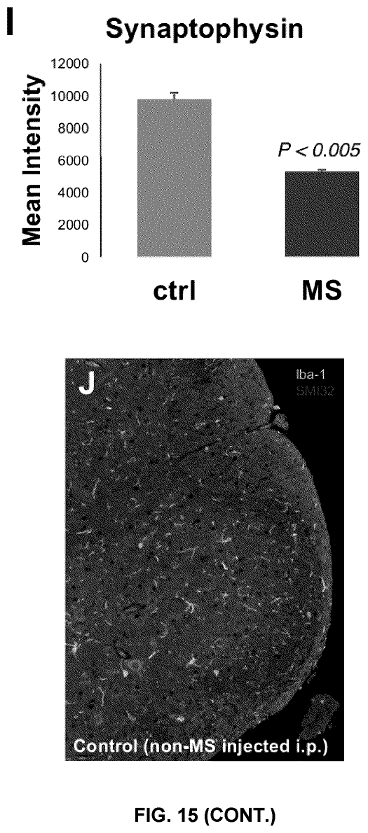

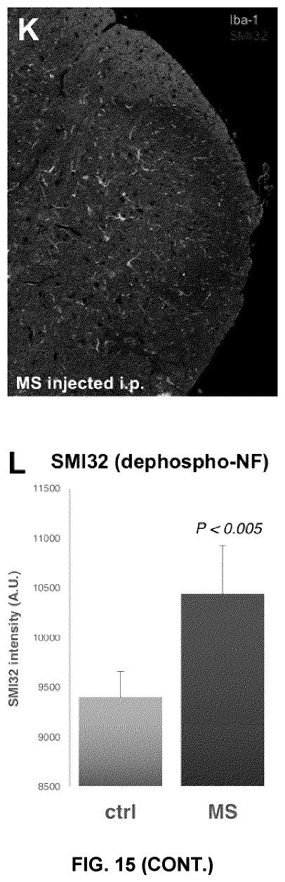

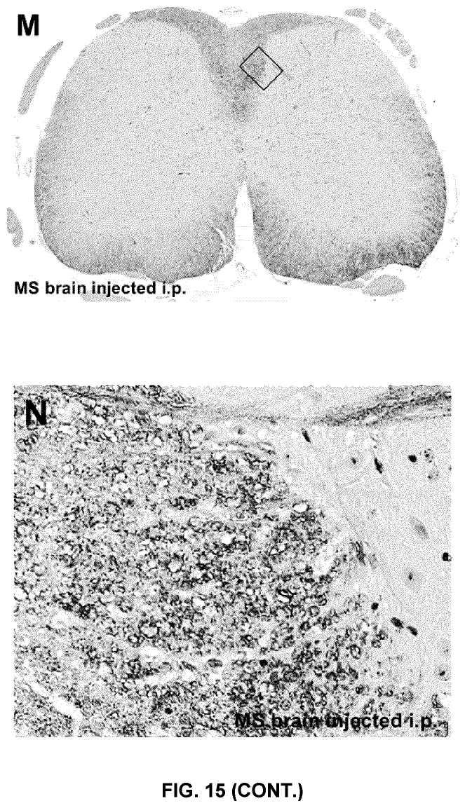

Additional experiments shown in FIG. 15 demonstrate the transmission of MS-like pathology following intraperitoneal injection of human MS spinal cord homogenate into mice including formic acid-resistant PrP deposits, myelin loss and synaptic damage not seen in controls. The observation that i.p. injection induces misfolded PrP accumulation in the dorsal aspects of the lumbar cord where afferents enter supports that the agent responsible for the transmission of the MS-like pathology tracks along peripheral fibers to invade the chord, similar to PrP prions.

Together, the data presented herein supports the position that MS is a weakly transmissible protease-sensitive prionopathy and that the etiological agent of MS is a misfolded prion conformer identified herein as PrP^{MS}.

In one embodiment, there is provided a method of identifying a subject having, or at risk of developing, multiple sclerosis (MS). In one embodiment, the method comprises determining the presence or absence of an abnormal prion protein conformer (PrP^{MS}) in a sample from the subject. In one embodiment, the presence of the abnormal prion protein conformer in the sample is indicative of the subject having MS or an increased risk of developing MS. In one embodiment, the PrP^{MS} conformer is absent in a control sample from a subject without MS. Optionally the control sample is matched for age, gender, ethnicity or genetic background with the subject who provides the sample.

As used herein, multiple sclerosis (MS) refers to a neurological disability characterized by formation of lesions or plaques in the central nervous system, inflammation, and/or the destruction of myelin sheaths of neurons leading to autonomic, visual, motor, and/or sensory problems. MS may be diagnosed on the basis of clinical presentation, medical imaging and/or laboratory testing. In one embodiment, MS may be diagnosed based on the McDonald criteria, including the dissemination of lesions in space and time. Optionally, a diagnosis of MS may include the use of magnetic resonance imaging (MRI) to show the existence of demyelinating lesions, that they occur in different areas of the nervous system and/or that they accumulate over time.

The term “subject” as used herein refers to any member of the animal kingdom. In one embodiment the subject is a mammal, such as a human. Optionally, the subject is a human presenting with one or more signs or symptoms of MS. In one embodiment, the subject is non-human animal, optionally a non-human primate. In one embodiment, the subject is a rodent, such as a mouse, hamster or a bank vole.

In one embodiment, determining the presence or absence of the abnormal prion protein conformer comprises detecting the presence or absence of the abnormal prion protein conformer in the sample.

Various methods may be used to detect the PrP^{MS} conformer described herein. For example, the PrP^{MS} conformer can be distinguished from cellular prion protein and/or prion proteins that are not associated with MS pathogenesis. In one embodiment, the PrP^{MS} conformer is detected in vitro or detected in vivo using a test subject. In one embodiment the PrP^{MS} conformer forms deposits of β-sheet rich amyloid.

The PrP^{MS} conformer may also be detected using biochemical techniques that distinguish between various forms of the PrP protein. For example, in one embodiment the PrP^{MS} conformer is detected by isolating or purifying prion proteins in a sample, subjecting the sample to digestion with a protease and detecting the protein fragments generated by protease digestion. In one embodiment, pronase-E digestion of the PrP^{MS} conformer generates smaller molecular weight fragments of prion protein that are resistant to further digestion, relative to pronase-E digestion of prion protein from a control sample from a subject without MS which almost completely digests the prion protein. In one embodiment, proteinase K or thermolysin digestion of the PrP^{MS} conformer generates smaller molecular weight fragments of prion protein that are resistant to further digestion, relative to proteinase k and/or thermolysin digestion of prion protein from a control sample from a subject without MS which almost completely digests the prion protein. In one embodiment, PrP^{MS} complexes are stabilized using Photo-Induced Cross-Linking of Unmodified Proteins (PICUP) prior to detection, such as by immunoassay, precipitation with PTA, and/or by spectral analysis. In one embodiment, the immunoassay comprises a Western blot to detect large molecular weight aggregates that differ in size from controls.

In one embodiment, the PrP^{MS} conformer may be detected in vivo using a test subject. As shown in the Examples, the PrP^{MS} conformer causes transmission of MS-like pathologies to a test subject when administered to the test subject. In one embodiment, the PrP^{MS} conformer causes transmission of MS-like pathologies to a test subject when injected intracerebrally into the test subject. In one embodiment, the test subject is a transgenic animal expressing human prion protein. In one embodiment, the test subject is a bank vole or mouse. In one embodiment, the test subject is a mouse, optionally a tg650 mouse or tga20 mouse. In one embodiment the presence or absence of PrP^{MS}conformer in the sample administered to the test subject is determined by detecting the presence or absence of lesions or plaques containing abnormal protein aggregates in the test subject. In one embodiment, the presence or absence of PrP^{MS} conformer in the sample administered to the test subject is determined by detecting MS-like pathologies in the test subject such as brain atrophy, amyloid plaque, demyelination and/or ventriculomegaly. In one embodiment, the presence of formic acid-resistant PrP aggregates in the test subject is indicative of the PrP^{MS} conformer in the sample.

As shown in the Examples, abnormal prion protein conformers associated with MS have been detected in brain tissue and cerebrospinal fluid. Furthermore, as shown in FIG. 9 blood samples from subjects with MS have been shown to carry aggregates of misfolded proteins. Accordingly, in one embodiment the sample is a brain tissue sample, a blood sample or a cerebrospinal fluid (CSF) sample from the subject. In one embodiment, the blood sample comprises blood elements selected from red blood cells, leukocytes, serum, exosomes, plasma and platelets. In one embodiment, the method comprises detecting the presence or absence of the PrP^{MS} conformer in blood elements. In one embodiment, the method comprises detecting the presence or absence of the PrP^{MS} conformer in leukocytes.

In one embodiment, the tissue sample is a brain tissue sample and the method comprises detecting the abnormal prion protein conformer in the brain tissue sample in extracellular space, around vessels, oligodendrocytes, myelin, neurons, cortex and/or white matter.

In one aspect, the presence or absence of the PrP^{MS} conformer in the sample is detected using spectral analysis. For example, the PrP^{MS} conformer may be detected using fluorescent spectroscopy and/or absorption spectroscopy and probes known to bind prion protein aggregates. In one embodiment the spectral analysis comprises:

(a) contacting the sample with a conformationally sensitive probe that binds to the abnormal prion protein conformer;

(b) generating a fluorescence emission spectrum or absorption spectrum of the conformationally sensitive probe bound to the abnormal prion protein conformer in the sample,

(c) comparing the fluorescence emission spectrum or absorption spectrum to one or more reference spectra.

In one embodiment, the reference spectra are representative of spectra from a subject or group of subjects known to have MS and correspondence between the reference spectra and the fluorescence emission spectrum or absorption spectrum is indicative that the subject has MS or an increased likelihood of developing MS. In another embodiment, the reference spectra are representative of spectra from a subject or group of subjects known not to have MS and correspondence between the reference spectra and the fluorescence emission spectrum or absorption spectrum is indicative that the subject does not have MS or an has a decreased likelihood of developing MS.

Conformationally sensitive probes useful for the spectral analysis of PrP conformers are known in the art. For example, in one embodiment the conformationally sensitive probe is selected from K114, Congo Red, a Congo Red derivative, X34, BSB, FSB, IMSB, Chrysamine-G, methoxy-X34, methoxy-X04, thioflavin-T, thioflavin-S, Pittsburgh compound B, thiazine red R, auramine-O, p-FTAA or a luminescent conjugated polythiophene (LCP) or luminescent conjugated oligothiophene (LCO) related to p-FTAA.

In another embodiment, the presence or absence of the PrP^{MS} conformer in the sample may be detected using binding agents such as antibodies and/or immunohistochemical methods.

In one embodiment, the method comprises fixation of the sample, optionally a blood sample or a tissue sample, using a fixative. In one embodiment, the fixative is formaldehyde, paraformaldehyde, glutaraldehyde, ethanol, methanol, acetone, or Hepes-glutamic acid-buffer mediated organic solvent protection effect (HOPE). In one embodiment, the fixing agent is formalin. In one embodiment, the fixed sample is heated, optionally to between about 80° C. and 98° C. for between about 10 and 30 minutes. In one embodiment, the sample is heated to 90-95° C. in citrate buffer for between about 10 and 20 minutes. In one embodiment, the fixed sample is treated with formic acid to selectively degrade PrP^{C}. In one embodiment, the fixed sample is treated with 70-100%, optionally 95-98% formic acid for between about 5 minutes and 30 minutes, optionally between 8 minutes and 12 minutes or about 10 minutes, followed by extensive washing in buffer. In one embodiment, the method comprises contacting the sample with an antibody that selectively binds to prion protein and detecting the antibody, optionally the 3F4 antibody.

In one embodiment, the sample from the subject may be treated to increase the amount of PrP^{MS} conformer in the sample prior to detection. For example, in one embodiment, the methods described herein include the use of protein-misfolding cyclic amplification (PMCA) prior to detecting the presence or absence of the PrP^{MS} conformer in the sample. In one embodiment, methods described herein include the use of photo-induced crosslinking methods (e.g. PICUP) prior to detecting the presence or absence of the PrP^{MS} conformer in the sample.

In one aspect, there is also provided a cell culture produced by contacting one or more cells with a composition comprising brain or spinal cord homogenate from a subject with MS and culturing the one or more cells. In one embodiment, there is provided a cell culture produced by contacting one or more cells with a composition comprising abnormal prion protein conformer (PrP^{MS}) and culturing the one or more cells. In one embodiment, the resulting PrP^{MS} cell culture contains one or more copies of the PrP^{MS} conformer resulting in the pathological aggregation of endogenous prion protein in the cell culture. In one embodiment, cells are brain cells and/or immune cells. In one embodiment, the cells are transgenic and/or recombinant cells that express or overexpression prion protein. In one embodiment, the cells express or overexpress human or bank vole prion protein.

As used herein the term “cell culture” refers to the maintenance and/or growth of one or more cells in a culture medium under controlled conditions. In one embodiment, the cells may be cultured as a layer on a substrate. In another embodiment, the cells may be cultured floating in a suspension. In one embodiment the cell culture comprises a plurality of cells of the same type or a plurality of cells of different types. As used herein the term “cell culture” includes tissue culture. For example, in one embodiment the cell culture is a culture of a plurality of cells from a tissue sample. In one embodiment the cell culture is a cerebellar tissue culture.

In one embodiment the cells are brain cells such as neurons, astrocytes, oligodendrocytes, oligodendrocyte precursor cells and/or microglia. In one embodiment, the cells are immune cells such as B cells and/or Follicular Dendritic cells, optionally alone or in combination with brain cells. In one embodiment, the cell culture comprises tissue culture. In one embodiment, the cell culture comprises central nervous system (CNS) tissue, optionally a slice culture of CNS tissue. In one embodiment, the cell culture comprises brain tissue. In one embodiment, the cell culture comprises cortical tissue, corpus callosum tissue, cerebellar tissue, ex vivo optic nerve tissue and/or spinal dorsal column/dorsal root ganglion (DRG) tissue. In one embodiment, the cells are transgenic cells. In one embodiment, the cells express or overexpress prion protein, optionally human prion protein. In one embodiment, the cells express or overexpress prion protein that form aggregates with human prion protein, such as prion protein from the bank vole. In one embodiment, the cells are from a stable cell line. In one embodiment, the cells are human glial (oligodendrocytic) cells that overexpress PrP. For example, in one embodiment the cells are from a huPrP-overexpressing MO3.13 cell line.

In one embodiment, a PrP^{MS} cell culture as described herein may be obtained by contacting one or more cells with a biological sample from a subject with multiple sclerosis. In one embodiment, the biological sample is a brain homogenate. Optionally, the biological sample is treated to increase the relative concentration of abnormally folded prion protein, such as by the use of phosphotungstic acid (PTA) to precipitate PrP^{MS} prior to contacting the one or more cells. In one embodiment, the biological sample is treated to stabilize the PrP^{MS} prion conformer prior to contacting the one or more cells, such as by Photo-Induced Cross-Linking of Unmodified Proteins (PICUP). In one embodiment, the cells used to generate the PrP^{MS} cell culture are recombinant cells. For example, in one embodiment the cells overexpress prion protein, optionally human prion protein or bank vole prion protein. In one embodiment, the cells used to generate the PrP^{MS} cell culture are cells from tg650 mice.

In one embodiment, the PrP^{MS} cell culture is obtained by contacting one or more cells with a PrP^{MS} cell culture as described herein or a preparation thereof. For example, in one embodiment the PrP^{MS} cell culture is obtained by contacting one or more cells with a composition comprising a PrP^{MS} cell culture that has been lysed and optionally contacted with phosphotungstic acid (PTA) to precipitate PrP^{MS} prior to contacting the one or more cells.

In one embodiment, the PrP^{MS} cell cultures described herein may be used in screening methods for identifying compounds suitable as diagnostic agents for the detection of MS and/or therapeutic agents for the treatment of MS.

In one embodiment, there is provided a method comprising:

(a) contacting a PrP^{MS} cell culture with a test compound; and

(b) detecting a biological effect of the test compound on the PrP^{MS} cell culture.

In one embodiment, the biological effect is a reduction in the presence of abnormal prion protein conformer and/or amyloid-like deposits in the PrP^{MS} cell culture relative to a control. Various methods, including but not limited to those described herein, may be used for detecting a biological effect resulting from the test compound on the PrP^{MS} cell culture. For example, in one embodiment detecting the biological effect of the test compound comprises histological, immunohistochemical and/or spectroscopic detection of abnormal prion protein conformer (PrP^{MS}) and/or amyloid plaques in the PrP^{MS} cell culture.

In one embodiment, a test compound that results in a decrease in the presence of PrP^{MS} conformer and/or amyloid plaques in the PrP^{MS} cell culture relative to a control is identified as a candidate therapeutic agent for the treatment of MS. In one embodiment, a test compound that results in a modification of cytotoxicity, cell maturity, cell viability and/or cytodegeneration in the PrP^{MS} cell culture relative to a control is identified as a candidate therapeutic agent for the treatment of MS. In one embodiment, a test compound that results in decreased cytotoxicity, increased cell maturity, increased cell viability and decreased cytodegeneration is identified as a candidate therapeutic agent for the treatment of MS.

In another aspect, there is provided a non-human animal model of multiple sclerosis (MS). In one embodiment, the non-human animal model of MS is produced by administering brain homogenate or spinal cord homogenate from a subject with MS to the non-human animal, optionally after passaging in one or more non-human animals. In one embodiment, the non-human animal model of MS is produced by administering abnormal prion protein conformer (PrP^{MS}) to a non-human animal. In one embodiment, the non-human animal develops one or more MS-like signs or symptoms after administration of the abnormal prion protein conformer. For example, in some embodiment, the non-human animal model of MS exhibits one or more MS-like signs or symptoms selected from the presence of amyloid plaques, demyelination, brain atrophy ventriculomegaly, and abnormal animal behavior.

The non-human animal may be a mammal, optionally a rodent such as a mouse, hamster or bank vole, or a non-human primate. Optionally, the non-human animal may be a transgenic animal that expresses or overexpresses prion protein. In one embodiment, the non-human may be a transgenic animal that expresses or overexpresses human prion protein. In one embodiment, the non-human animal is a tg650 mouse

Different methods may be used to administer an effective dose of PrP^{MS} conformer to a non-human animal to produce an animal model of MS. In one embodiment, a composition comprising PrP^{MS} conformer is administered to the non-human animal by intracerebral or intraperitoneal injection. The composition comprising PrP^{MS} conformer may be brain homogenate from a subject with MS. Alternatively, the composition may be a PrP^{MS} cell culture or preparation thereof such as a PrP^{MS} cell culture treated to increase the concentration of PrP^{MS} conformers.

In one embodiment, the non-human animal models of MS described herein may be used in screening methods for identifying compounds suitable as diagnostic agents for the detection of MS and/or therapeutic agents for the treatment of MS.

In one embodiment there is provided a method comprising:

(a) administering a test compound to a non-human animal model of MS as described herein; and

(b) detecting a biological effect of the test compound on the non-human animal model.

The biological effect may be a reduction in the presence of abnormal prion protein conformer and/or amyloid-like deposits in the non-human model organism relative to a control. Various methods, including but not limited to those described herein, may be used for detecting a biological effect resulting from the test compound on the non-human model of MS.

For example, in one embodiment detecting the biological effect of the test compound comprises histological, immunohistochemical and/or spectroscopic detection of abnormal prion protein conformer (PrP^{MS}) and/or amyloid plaques in a sample from the non-human model of MS.

In one embodiment, the biological effect is an increase or decrease in one or more signs or symptoms of MS in the model animal. For example, in one embodiment, the signs or symptoms of MS are selected from the presence of amyloid plaques, brain atrophy, demyelination, ventriculomegaly, abnormal animal behavior and/or motor/pain perception/cognitive/visual abnormalities. In one embodiment, the biological effect is a reduction in the presence of PrP^{MS} conformer and/or amyloid-like deposits in the non-human model animal relative to a control animal. In one embodiment, the biological effect is demyelination and/or degeneration, optionally measured through Magnetic Resonance Imaging (MRI), Computerized Tomography (CT) and/or Micro/Nano Computed Tomography (micro-CT and/or nano-CT).

In one embodiment, a test compound that results in a decrease in the presence of PrP^{MS} conformer and/or amyloid plaques or a decrease in one or more signs or symptoms of MS in the non-human animal model of MS relative to a control is identified as a candidate therapeutic agent for the treatment of MS.

In one aspect, the inventors have surprisingly determined that subjects with multiple sclerosis (MS) exhibit abnormal deposits of prion protein (PrP) and that MS pathology is associated with a progressive transmissible prion protein folding disorder. Agents that reduce a level and/or inhibit the formation of the abnormally folded prion protein may therefore be useful for the prevention or treatment of MS.

Without being limited by theory, it is believed that MS is caused by misfolding of normal prion protein (PrP^{C}) into an aggregation-prone conformer, PrP^{MS}, which accumulates in the brain of MS patients and causes dysregulation of myelinic GluRs and non-immune demyelination. The accumulation of pathological prion may induce chronic dysregulation of the axo-myelinic synapse culminating in progressive disease. Previously it was not known that abnormal PrP could be detected in subjects with MS nor has there been any biological data demonstrating disease transmission consistent with MS being a prionopathy. In contrast, a commonly accepted view is that MS is a primary autoimmune disease associated with genetic and/or environmental risk factors.

Together, the data presented herein supports the position that MS is a weakly transmissible protease-sensitive prionopathy and that the etiological agent of MS is a misfolded prion conformer identified herein as PrP^{MS}. Agents that reduce the levels of abnormally folded prion protein and/or inhibit the formation of abnormally folded prion protein may therefore be useful for the prevention or treatment of MS. In one embodiment, the agents that reduce and/or inhibit the formation of PrP^{MS} are listed in Table 1.

TABLE 1
Agents for the prevention or treatment of MS caused by PrP^{MS}
Agent

Quinacrine
Pentosan polysulphate (PPS)
Tetracycline antibiotics, including doxycycline and minocycline
Astemizole
Tacrolimus (FK-506)
Fluorescent Amyloid dyes (e.g. K114, Congo Red, a Congo Red
derivative, X34, BSB, FSB, IMSB, Chrysamine-G, methoxy-X34,
methoxy-X04, thioflavin-T, thioflavin-S, Pittsburgh compound B,
thiazine red R, auramine-O, pFTAA, or a luminescent conjugated
polythiophene (LCP) or luminescent conjugated oligothiophene
(LCO) related to p-FTAA.
Anle138b
Antibodies that bind to PrP^{MS}
Molecular modifications of the agents listed herein with the
purpose of improving bioavailability, safety or crossing of the
blood brain barrier

In one embodiment, the agent is quinacrine and is for the treatment and/or prevention of MS. Korth et al. [121] (Proc Natl Acad Sci USA. 2001 Aug. 14; 98(17):9836-41, hereby incorporated by reference) reported that quinacrine inhibited the formation of pathogenic prions (PrP^{SC}) in cultured cells. In one embodiment, quinacrine may therefore be useful for reducing and/or preventing the formation of PrP^{MS} conformers.

In one embodiment, the agent is pentosan polysulphate (PPS) and is for the prevention of MS. Doh-ura et al. [122] (J Virol. 2004 May; 78(10):4999-5006, hereby incorporated by reference) showed that intraventricular application of PPS prolonged the incubation period in transgenic mice infected with scrapie agent. In one embodiment, PPS may therefore be useful for reducing and/or preventing the formation of PrP^{MS} conformers.

In one embodiment, the agent is a tetracycline antibiotic, such as doxycycline or minocycline, and is for the prevention of MS. De Luigi et al. [123] (PLoS One. 2008 Mar. 26; 3(3):e1888, hereby incorporated by reference) described the efficacy of tetracyclines in peripheral and intracerebral prion infection. In one embodiment, tetracyclines may therefore be useful for reducing and/or preventing the formation of PrP^{MS} conformers.

In one embodiment, the agent is astemizole and is for the treatment and/or prevention of MS. Karapetyan et al. [124] Proc Natl Acad Sci USA. 2013 Apr. 23; 110(17):7044-9, hereby incorporated by reference) identified astemizole as reducing cell-surface PrP and inhibiting prion replication in neuroblastoma cells. In one embodiment, astemizole may therefore be useful for reducing and/or preventing the formation of PrP^{MS} conformers.

In one embodiment, the agent is tacrolimus (FK-506) and is for the prevention of MS. Karapetyan et al. [124] Proc Natl Acad Sci USA. 2013 Apr. 23; 110(17):7044-9, hereby incorporated by reference) identified tacrolimus as reducing cell-surface PrP and inhibiting prion replication in neuroblastoma cells. In one embodiment, tacrolimus may therefore be useful for reducing and/or preventing the formation of PrP^{MS} conformers.

In one embodiment, the agent is a fluorescent amyloid dye and is for the treatment and/or prevention of MS. Herrmann et al. [125] (Sci Transl Med. 2015 Aug. 5; 7(299):299ra123, hereby incorporated by reference) used structure-based drug design to identify polythiophenes as antiprion compounds. Fluorescent amyloid dyes are known to bind to pathogenic PrP conformers as demonstrated herein and are therefore expected to be useful for reducing and/or preventing the formation of PrP^{MS} conformers

In one embodiment, the agent is Anle138b and is for the treatment and/or prevention of MS. Wagner et al. [126] (Acta Neuropathol. 2013 June; 125(6):795-813, hereby incorporated by reference) described Anle138b as an aggregation inhibitor for the therapy of neurodegenerative diseases such as prion disease. In one embodiment, Anle138b may therefore be useful for reducing and/or preventing the formation of PrP^{MS} conformers.

In one embodiment, antibodies that selectively bind to PrP^{MS} may be useful for reducing and/or preventing the formation of PrP^{MS} conformers. In one embodiment, there is provided an antibody that selectively binds to PrP^{MS}. The term “antibody” as used herein is intended to include monoclonal antibodies, polyclonal antibodies, antibody fragments, chimeric antibodies, single domain antibodies, humanized antibodies, human antibodies, single chain antibodies and bispecific antibodies. The antibody may be from recombinant sources and/or produced in transgenic animals. The term “antibody fragment” as used herein is intended to include Fab, Fab′, F(ab′)2, scFv, dsFv, ds-scFv, dimers, minibodies, diabodies, and multimers thereof and bispecific antibody fragments. Antibodies can be fragmented using conventional techniques. For example, F(ab′)2 fragments can be generated by treating the antibody with pepsin. The resulting F(ab′)2 fragment can be treated to reduce disulfide bridges to produce Fab′ fragments. Papain digestion can lead to the formation of Fab fragments. Fab, Fab′ and F(ab′)2, scFv, dsFv, ds-scFv, dimers, minibodies, diabodies, bispecific antibody fragments and other fragments can also be synthesized by recombinant techniques.

In one embodiment, there is provided a method for the treatment or prevention of multiple sclerosis (MS) comprising administering to a subject an effective amount of agent that reduces and/or inhibits the formation of PrP^{MS} conformers. Also provided is the use of an effective amount of agent that reduces and/or inhibits the formation of PrP^{MS} conformers for the treatment or prevention of multiple sclerosis (MS) in a subject in need thereof. In one embodiment, the method further comprises identifying the subject as having, or at risk of development, MS as described herein, prior to administering to the subject an effective amount of agent that reduces and/or inhibits the formation of PrP^{MS} conformers.

As used herein, the term “effective amount” refers to an amount of an agent that is sufficient to produce a desired effect, which can be a therapeutic, protective and/or beneficial effect. The effective amount will vary with the age, general condition of the subject, the severity of the condition being treated, the particular agent used or administered, the duration of the use or treatment, the nature of any concurrent treatments, the pharmaceutically acceptable carrier used, and like factors within the knowledge and expertise of those skilled in the art. As appropriate, an “effective amount” in any individual case can be determined by one of ordinary skill in the art by reference to the pertinent texts and literature and/or by using routine experimentation. (See, for example, Remington, The Science and Practice of Pharmacy (20th ed. 2000)).

The terms “treat,” “treating” or “treatment” as used herein refer to any type of action that imparts a modulating effect, which, for example, can be a beneficial and/or therapeutic effect, to a subject afflicted with a condition, disorder, disease or illness, including, for example, improvement in the condition of the subject (e.g., in one or more symptoms), delay in the progression of the disorder, disease or illness, prevention or delay of the onset of the disease, disorder, or illness, and/or change in clinical parameters of the condition, disorder, disease or illness, etc., as would be well known in the art. The terms “treat,” “treating” or “treatment” as used herein also mean administering to a subject or the use of a therapeutically effective amount of the agents described herein and may consist of a single administration or use, or alternatively comprise a series of applications or uses.

As used herein, and as well understood in the art, “treatment” or “treating” is also an approach for obtaining beneficial or desired results, including clinical results. Beneficial or desired clinical results can include, but are not limited to, alleviation or amelioration of one or more symptoms or conditions, diminishment of extent of disease, stabilized (i.e. not worsening) state of disease, delay or slowing of disease progression, amelioration or palliation of the disease state, and remission (whether partial or total), whether detectable or undetectable. “Treatment” can also mean prolonging survival as compared to expected survival if not receiving treatment. Further any of the treatment methods or uses described herein can be formulated alone or for contemporaneous administration with other agents or therapies.

The terms “prevent,” “preventing,” and “prevention” and like terms are used herein to include imparting any level of prevention or protection which is of some benefit to a subject, such that there is a reduction in the incidence and/or the severity of the disease in a treated subject, regardless of whether the protection or reduction in incidence and/or severity is partial or complete.

For example, the “treatment or prevention of MS” may include alleviation or amelioration of one or more signs or symptoms of MS such as amyloid plaques, brain atrophy, demyelination, microgliosis, proliferation of oligodendrocyte precursors, ventriculomegaly and abnormal behavior. In one embodiment, the “treatment or prevention of MS” may include extending the time between relapses for a subject.

In one embodiment, the agents described herein may be for use, formulated for use, or administered to a subject in need thereof. In one embodiment, the agents described herein are formulated or modified to cross the blood brain barrier.

Conventional procedures and ingredients for the selection and preparation of suitable formulations are described, for example, in Remington's Pharmaceutical Sciences (2003—20th edition) and in The United States Pharmacopeia: The National Formulary (USP 24 NF19) published in 1999.

Use or administration can be effected in one dose, continuously or intermittently (e.g., in divided doses at appropriate intervals) throughout the course of treatment. Methods of determining the most effective means and dosage of use or administration are well known to those of skill in the art and will vary with the formulation used for therapy, the purpose of the therapy, the target cell(s) being treated, and the subject being treated. Single or multiple uses or administrations can be carried out with the dose level and pattern being selected by the treating physician, veterinarian, or clinician.

Routes of administration include, but are not limited to, oral (e.g., by ingestion); buccal; sublingual; transdermal (including, e.g., by a patch, plaster, etc.); transmucosal (including, e.g., by a patch, plaster, etc.); intranasal (e.g., by nasal spray, drops or from an atomiser or dry powder delivery device); ocular (e.g., by eyedrops); pulmonary (e.g., by inhalation or insufflation therapy using, e.g., an aerosol, e.g., through the mouth or nose); rectal (e.g., by suppository or enema); vaginal (e.g., by pessary); parenteral, for example, by injection, including subcutaneous, intradermal, intramuscular, intravenous, intraarterial, intracardiac, intrathecal, intraspinal, intracapsular, subcapsular, intraorbital, intraperitoneal, intratracheal, subcuticular, intraarticular, subarachnoid, and intrasternal; by implant of a depot or reservoir, for example, subcutaneously or intramuscularly. In one embodiment, the route of administration is oral (e.g., by ingestion). In another embodiment, the route of administration is parenteral (e.g., by injection).

Also provided is a method for reducing and/or inhibiting the formation of PrP^{MS} conformers in CNS cells or tissue. In one embodiment, the method comprises contacting CNS cells or tissue with an agent listed in Table 1. Optionally, the CNS cells or tissue are in vivo or in vitro. In one embodiment, the CNS tissue comprises cortical tissue, corpus callosum tissue, cerebellar tissue, optic nerve tissue and/or spinal dorsal column/dorsal root ganglion (DRG) tissue. In one embodiment, the CNS cells are brain cells or immune cells. In one embodiment, the brain cells are neurons, astrocytes, oligodendrocytes, oligodendrocyte precursor cells and/or microglia. In one embodiment, the immune cells are B cells and/or Follicular Dendritic cells.

The above disclosure generally describes the present invention. A more complete understanding can be obtained by reference to the following specific examples of certain embodiments of the invention.

### EXAMPLES

### Example 1: Detection of Prion Protein and Amyloid Deposits in MS

Using a conformationally-sensitive fluorescent amyloid probe (pFTAA) and spectral microscopy, widespread amyloid deposits were detected in cortex, white matter and around blood vessels in human MS brain (FIG. 1CD, controls 1AB). This observation demonstrates that the MS-specific aggregates are β sheet-rich pathological amyloids that are potentially toxic.

Comparing the spectra of normal brain and mature amyloid from MS brain (FIG. 1F), distinct spectral features are observed. Using proprietary software to score an image based on the spectra, quantifiable differences between control and PPMS brain can be observed (FIG. 1E).

The background spectral shift in shown FIG. 1 is strong evidence for a widespread increased deposition of toxic PrP oligomers, which would explain the widespread but subtle abnormalities observed in “normal-appearing” MS gray and white matter.

### Example 2: Investigations of PrP^{MS} in Transgenic Mice Expressing Human PrP

Transmission experiments were performed using the tg650 mouse as the host, which overexpresses human PrP [68,69]. Six mice were injected intracerebrally with homogenate of human brain from a secondary progressive MS patient. A control group of tg650 mice was injected (n=5+2 uninjected age-matched) with non-MS human brain (terminal lung disease). The mice were followed by serial MRI brain imaging. After ≈1 year of incubation, MS-inoculated mice developed obvious brain atrophy and ventriculomegaly (FIG. 2), a common finding in advanced MS[70]. Importantly, none of the controls exhibited any significant pathological change. Very sensitive methods for analyzing quantitative T2 MRI data were developed (FIGS. 2E-J). This approach uncovered subtle pathology in MS-inoculated mouse brain without anatomical T2 differences, indicating that the rate of transmission was higher than initially appreciated. Subsequent passage data (brain from affected mouse as above inoculated into naïve tg650 mouse), also showed pathology relative to controls (not shown). It is extremely improbable that some other human agent, a virus (with no evidence of encephalitis), or an adoptive transfer of autoimmunity, was responsible for the pathology at primary inoculation, and that it would continue to induce pathology almost 2 years later after passaging.

In addition, inoculated mouse brains frequently showed abnormal plaque-like PrP deposits. As shown in FIGS. 2, 3, 7 and 8, MS-inoculated mouse brains showed pathology affecting the corpus callosum—a structure invariably affected in human MS [71]—with atrophy, vacuolar change, demyelination and deposition of PrP “plaques” as well as atrophy in the cortex and cerebellum. Notably, histopathology showed very little lymphocytic inflammation, in striking contrast to relapsing-remitting MS plaques and mouse EAE, strongly arguing against adoptively transferred immune elements.

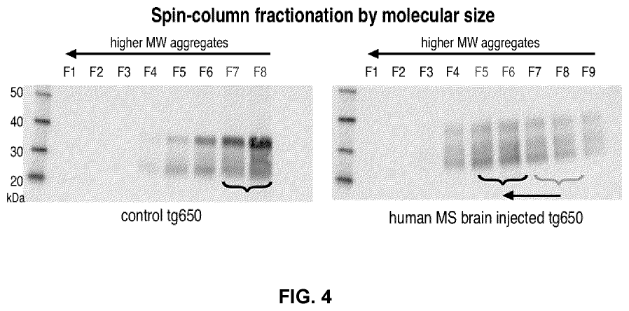

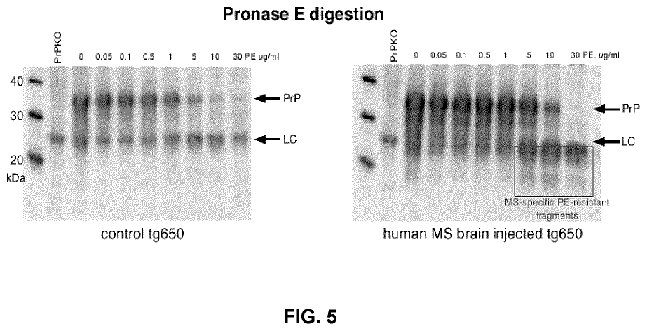

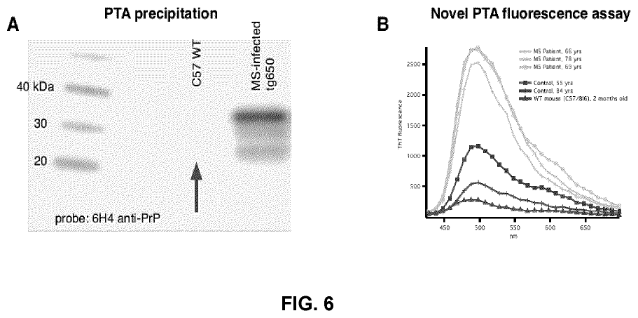

Additional biochemical investigations into abnormal prion protein from tg650 mice were performed. Spin column fractionation revealed that MS-inoculated tg650 brain exhibited a shift to higher molecular weight complexes indicating an abnormally-aggregated PrP (FIG. 4). Using pronase-E digestion, smaller MW fragments of PrP were detected from MS-injected mouse brain but never from controls (FIG. 5). Smaller undigested fragments of PrP are characteristic of many prionopathies [64,65,72]. PTA precipitation (a technique used to selectively precipitate abnormally aggregated PrP [73,74]) showed substantial amounts of aggregated PrP in infected mouse brain, but none in naïve mice (FIG. 6A). An assay coupling fluorescence and PTA precipitation, showed strongly positive PrP aggregates in MS brain, not seen in controls (FIG. 6B).

### Example 3: Detection of MS Aggregated Protein in Leukocytes

Blood samples were obtained from 6 patients with MS and 5 non-MS controls including two patients with Alzheimer's disease. Leukocytes were isolated and stained with the fluorescent amyloid probe pentameric formyl thiophene acetic acid (pFTAA).

As shown in FIG. 9, the spectral analysis of leukocytes was able to detect 5 out of 6 patients with MS and none of the non-MS controls were identified as having MS. The white arrows in FIG. 9A show the detection of protein aggregates in leukocytes from patients with MS.

The spectra of pFTAA in presence of MS PrP amyloid is specific to MS (FIG. 9B). Using the basis spectra from FIG. 9B, 5 of 6 MS patients had significant disease scores, indicating the presence of MS specific amyloid, possibly consisting of PrP. Alzheimer's patients were included in the analysis, and even though they also accumulate amyloid, it is of a different protein (Amyloid-β) and thus they do not score highly on this test.

### Example 4: Transmission to Mice Overexpressing Human PrP

Experiments are performed showing the primary inoculation from human brain to transgenic mouse, (termed “1st passage”). Homogenates from frozen MS brain are prepared in sterile PBS and sonicated to break up larger aggregates (and any immune cells) to increase infectivity. 30 μl of 1% homogenate is injected intracerebrally into 12 mice; in parallel, 100 μl of 10% homogenate is injected intraperitoneally into 12 mice from each of 9 patients. Scrapie prions are known to be transmissible after oral ingestion and absorption from gut. Subtle myelopathy may develop in i.p. injected mice as the agent is transported into the cord from the peritoneal cavity. Controls from 12 non-MS patients are included to determine if detected changes are MS-specific.

Identical experiments are done using tga20 (murine prion overexpressor): it is predicted that human PrP^{MS} will cross this species barrier with difficulty if at all. Mice may be incubated for extended periods to confirm this negative control. It is possible that tga20 may become “infected”, as the species barrier may not be absolute; this would not negate our hypothesis. Therefore the following controls are used: same experiments as above but into PrP null and wild type mice. Scrapie transmission depends on expression of endogenous prion protein and PrP nulls are resistant to infection [87]. Inoculation of wild type mice (expressing normal levels of murine prion) will exclude a non-specific pathological effect of injecting human brain into mouse. If human MS transmits to tg650 (human PrP overexpressor), but not to tga20 (mouse PrP overexpressor), this will provide very strong evidence for: a) prion as the pathogenic agent in MS, and b) specificity of PrP^{MS} implying a unique human conformer. While selective transmission to human PrP-overexpressing mice, but not to WT with murine PrP or to PrP nulls, would provide strong evidence for a prion being the responsible agent, the possibility would still exist that a transmissible agent that was not a prion was responsible, and that the overexpressors were simply more susceptible to “infection”. To address this possibility, homogenates of human MS brain, and tg650 mouse brain successfully infected after 1st passage, will be immunodepleted of PrP using magnetic beads. No disease in mice injected with immunodepleted homogenate is expected, providing unequivocal evidence that prion protein was involved in the transmission. Equally important, these controls provide strong evidence against an unexpected infectious pathogen (e.g. a virus), or an adoptive immune transfer (e.g. activated T cells, antibodies), being responsible for transmission.

MRI: high-field MRI is used to non-invasively track development of pathology. Groups of animals (≈20 mice per month on average) are scanned using anatomical and quantitative T2 sequences. The former gives a complete “z-stack” through the entire brain, whereas the latter yields reliable data on pathological changes (mainly cellular edema/vacuolation/spongiform change) using our quantitative analysis techniques (FIG. 2). A standard qT2 slice at the level of the hippocampus and a second through the cerebellum are acquired at each session. Animal groups are rotated so that each subject will be serially scanned approximately every 3 months.

Passaging: A cardinal feature of prion diseases is shorter latency to disease and crossing of species barriers with repeated passaging, that is, harvesting previously inoculated brain after the first human-to-mouse injection and re-inoculating into naïve mouse [88-90]. This intriguing phenomenon is thought to be due to rising titres and conformational adaption of the pathogenic prion to the host species [90,91]. Brain is harvested from significantly affected (by MRI) tg650 mice, and homogenates are injected i.c. and i.p. back into naïve tg650 mice. Animals are followed by MRI, and then histology. A more rapid pathology is observed with 1st passage; this is repeated at least twice more in an attempt to accelerate pathology which eventually reaches a plateau [88]. Passaging provides further evidence for an adaptive prion as the pathogenic agent. Moreover if the latency to pathology can be reduced from ≈1 year to several months, this passaged brain material represents an invaluable reagent for creating a unique in vivo model of progressive MS.

Histology: Examination of fixed tissue with histology and immunochemistry is used to study pathological details typical of MS pathology. Techniques and probes include titrated digestion and amyloid probe staining to detect partially protease resistant, misfolded β sheet-rich PrP aggregates. Examination of the spinal cord for demyelination, axonal injury and amyloid deposition after i.p. injection provides strong evidence that the pathogenic agent entered via afferents into the cord as is described in traditional scrapie [85], if cord pathology is observed in these and not in i.c. injected mice.

Biochemistry: Experiments are conducted to further characterize the PrP^{MS} conformer. Brain homogenates from wild type, age-matched tg650 and inoculated tg650 (1^{st} 2^{nd} and higher passage) are digested with various concentrations of pronase E (0-30 μg/ml) and run on Westerns, and probed with 6H4. Other PrP antibodies (3F4; 1E4 the latter being particularly good at detecting digested VPSPr's [93]) are also tested because cleavage at certain sites may variably affect immunoreactivity of the smaller remaining fragments. Passaging may increase the detectable titres of partially resistant cleavage products as is seen with scrapie [51,81,94]. The protocol is also performed on human MS brain samples to detect an abnormal PrP conformer. The experiments are repeated using thermolysin (0-150 μg/ml), another protease that degrades PrPC while preserving both PK-sensitive and PK-resistant misfolded PrP isoforms [95,96].

Phosphotungstic acid (PTA) precipitation is very effective for eliminating normal un-aggregated proteins (including PrPC) and greatly improving detection of abnormally folded pathogenic PrP, regardless of protease sensitivity [92]. Control and inoculated tg650 mouse and human MS brain samples as above are precipitated using NaPTA ([92,97,98] and Fig. S1C). Additional pronase E digestion is tested to further isolate abnormal PrP. Pellets are resuspended, run on Westerns and probed with 6H4, 3F4 and 1 E4. This method should greatly increase ability to distinguish between normal PrPC vs. inoculated tg650 brain. Given the ability of PTA to concentrate and purify abnormal PrP by over 2 orders of magnitude while retaining its infectivity [92], these precipitates are also used for re-inoculation, which may greatly accelerate onset of pathology.

### Example 5: Evidence of a MS Prionopathy in Human Material

Sections from MS brain and cord, from 6 SP and 6 PPMS patients are analyzed using hydrolytic and proteolytic techniques [106-108] to reveal abnormally folded/aggregated PrP over the normal PrPC background. Human MS and control brain samples are i) heated to 96° C. in citrate buffer, treated with formic acid (99%×10min), both of which reduce PrPC staining, increasing contrast of abnormal PrP signal [46]. Exposure times are titrated to eliminate normal PrPC signal in control while maximizing abnormal PrP staining in MS brain. Preferred PrP antisera include 3F4 and 6H4 [109,110]. Titres are adjusted to minimize control, and maximize MS-related staining. Sections (either before or after heating/acid hydrolysis) are exposed to protease at varying concentrations.

Demonstration of partially protease-resistant PrP in MS brain, but not in controls, provides very strong evidence of a misfolded and likely pathogenic prion protein.

### Example 6: MS Brain and Spinal Cord Transmits Pathology to Humanized Transgenic Mice

### Materials and Methods

Human brain and spinal cord homogenates were prepared from controls (non-neurological death, Alzheimer's and Lewy body diseases, and chronic encephalitis) and from 10 primary and secondary progressive MS patients, sonicated and 30 μl injected intracerebrally under anesthesia into tg650 human prion protein over-expressing mice (MM at codon 129). Animals were imaged using anatomical and quantitative T2 sequences on a Bruker 9.4T animal MRI. At termination, mouse brains were either frozen for biochemistry or fixed in formalin and paraffin embedded for histology and immunohistochemistry. Pathological PrP aggregates were immunodetected (3F4 antibody) after heat antigen retrieval and formic acid treatment to differentiate from PrP^{C}. Immunofluorescence was performed using standard techniques. All error bars are s.e.m. P values by non-parametric Wilcoxon rank test.

### Results

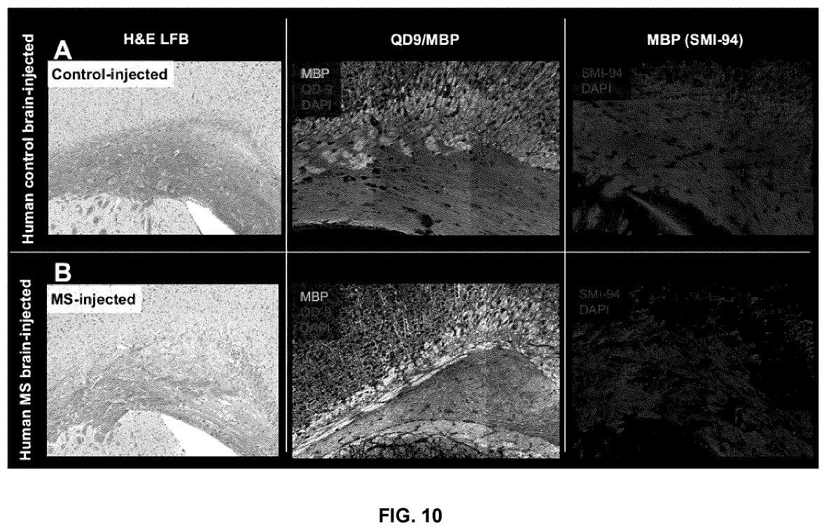

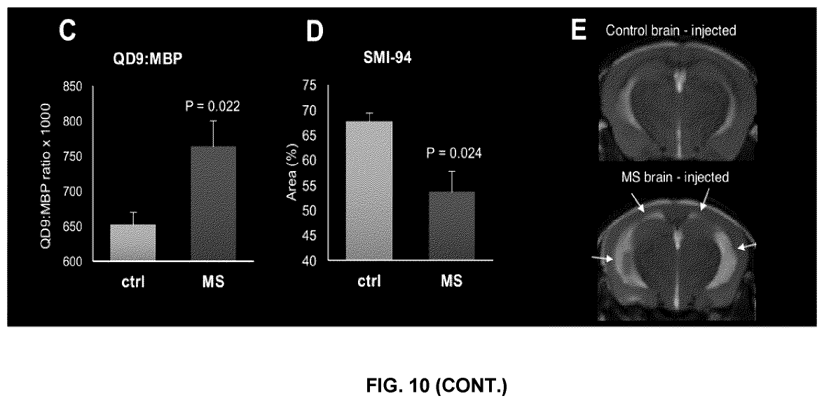

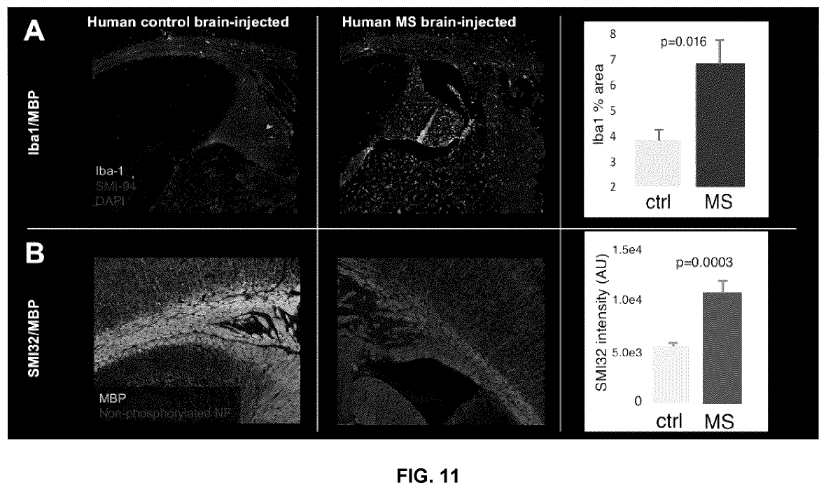

As shown in FIG. 10, injection of human MS brain homogenate induced demyelination of the corpus callosum in tg650 mice overexpressing human prion protein. Immunofluorescence micrographs staining for Iba-1 and SMI32 also indicated microglial activation and axonal injury that are patterns characteristic of human MS (FIG. 11).

In addition to the molecular pathologies observed in MS brain injected mice, injected mice exhibited abnormal behaviours not seen in controls. Specifically, MS brain-injected mice showed both deficits in a water maze test and as well as decreased exploratory behaviour/more anxiety (FIG. 12)

As shown in FIG. 13, immunohistochemical staining for prion protein in brain sections using the 3F4 antibody showed formic acid resistant deposits reminiscent of proteinase-resistant prionopathy. These “PrP^{MS}” deposits were observed in extracellular brain parenchyma and in perivascular spaces. Remarkably, abnormal PrP^{MS} deposits were also observed after multiple passaging in tg650 mice.

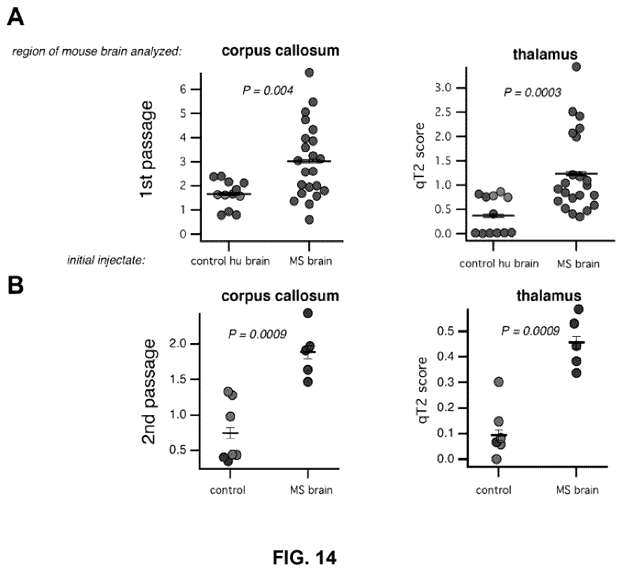

Quantitative T2 MRI analysis of 1^{st} passage mice injected i.c. with MS brain homogenate demonstrated pathology in the corpus callosum and thalamus also seen in 2^{nd} passage mice (FIG. 14).

Human MS spinal cord homogenate was also demonstrated to result in the transmission of pathology. As shown in FIG. 15, intraperitoneal injection of MS homogenate resulted pathology not seen in controls. Notably, mice injected with MS spinal cord homogenate i.p. exhibited pronounced FA-resistant PrP deposits. Furthermore, significant myelin loss and synaptic damage were observed in MS-injected lumbar cord. A disease-associated factor in MS homogenate therefore appears able to invade the CNS after i.p. inoculation, in a region-specific fashion, and in a manner similar to scrapie prions.

### Discussion

MS exhibits prominent features of both recurrent autoimmune inflammation and progressive degeneration. While current therapies are highly successful at mitigating the former, even potent immune suppression shows little benefit against progression. Together with reports of very early MS lesions showing profound demyelination with little inflammation, this suggests that MS may be a primary degenerative disorder, with the commonly seen inflammatory overlay an important, but secondary, phenomenon. The results presented herein are consistent with the hypothesis that MS, like other degenerative disorders, may be a transmissible protein misfolding disorder. One component might involve a disease-specific conformer of human prion protein. However, while seemingly transmissible across multiple passages, this conformer does not exhibit properties of more conventional transmissible spongiform encephalopathies, such as proteinase K resistance or abnormalities using conformationally-dependent immunoassay (data not shown).

Without being limited by theory, the MS-specific agent(s) may target the myelin and oligodendrocytes, possibly by chronically dysregulating the axo-myelinic synapse [120]. Chronic release of antigenic myelin debris, coupled with a dysregulated immune system, together may result in the characteristic inflammatory relapsing-remitting MS phenotype. In contrast, the same underlying degenerative process, but with a weaker autoimmune predilection, would instead promote a less inflammatory, more degenerative primary progressive course.

As set out above, human MS brain and spinal cord homogenates have been demonstrated to transmit pathology (MRI, histology, behavior) when injected either intracerebrally or intraperitoneally into human prion protein overexpressing mice. Remarkably, transmission of pathology persisted over multiple (4) passages. Histologically, MS-inoculated mouse brains exhibited FA resistant PrP deposits indicative of an abnormal conformer, typical of protein misfolding degenerations. The results presented herein are consistent with the hypothesis that an abnormal conformer of human prion protein (“PrP^{MS}”) plays an important role in MS pathogenesis. Variable degrees of dysimmunity (determined by e.g. environment, genetics), convolved with a chronic cytodegeneration, can provide a unifying hypothesis to explain the broad spectrum of clinical MS phenotypes.

While the present disclosure has been described with reference to what are presently considered to be the preferred examples, it is to be understood that the disclosure is not limited to the disclosed examples. To the contrary, the disclosure is intended to cover various modifications and equivalent arrangements included within the spirit and scope of the appended claims.

All publications, patents and patent applications are herein incorporated by reference in their entirety to the same extent as if each individual publication, patent or patent application was specifically and individually indicated to be incorporated by reference in its entirety.

### REFERENCES

1. Multiple Sclerosis International Federation. Atlas of MS 2013. http://www.msif.org/wp-content/uploads/2014/09/Atlas-of-MS.pdf
2. Lassmann, H. (2005) Multiple sclerosis pathology: evolution of pathogenetic concepts. Brain Pathol 15, 217-222
3. Peterson, J. W. and Trapp, B. D. (2005) Neuropathobiology of multiple sclerosis. Neurol Clin 23, 107-29, vi-vii
4. Geurts, J. J. et al. (2012) Measurement and clinical effect of grey matter pathology in multiple sclerosis. Lancet Neurol 11, 1082-1092
5. Confavreux, C. and Vukusic, S. (2006) Accumulation of irreversible disability in multiple sclerosis: from epidemiology to treatment. Clin. Neurol. Neurosurg. 108, 327-332
6. Nylander, A. and Hafler, D. A. (2012) Multiple sclerosis. J Clin Invest 122, 1180-1188
7. Steinman, L. (2014) Immunology of relapse and remission in multiple sclerosis. Annu Rev Immunol 32, 257-281
8. Sawcer, S., Franklin, R. J. and Ban, M. (2014) Multiple sclerosis genetics. Lancet Neurol 13, 700-709
9. Giovannoni, G. and Ebers, G. (2007) Multiple sclerosis: the environment and causation. Curr Opin Neurol 20, 261-268
10. Belbasis, L. et al. (2015) Environmental risk factors and multiple sclerosis: an umbrella review of systematic reviews and meta-analyses. Lancet Neurol
11. Frischer, J. M. et al. (2009) The relation between inflammation and neurodegeneration in multiple sclerosis brains. Brain 132, 1175-1189
12. Stys, P. K. (2011) The axo-myelinic synapse. Trends Neurosci. 34, 393-400
13. Micu, I. et al. (submitted) The molecular physiology of the axo-myelinic synapse. Elife
14. Trapp, B. D. et al. (1998) Axonal transection in the lesions of multiple sclerosis. N. Engl. J. Med. 338, 278-285
15. Seewann, A. et al. (2009) Diffusely abnormal white matter in chronic multiple sclerosis: imaging and histopathologic analysis. Arch. Neurol. 66, 601-609
16. Filippi, M. et al. (2012) Association between pathological and MRI findings in multiple sclerosis. Lancet Neurol 11, 349-360
17. Rodriguez, M. and Scheithauer, B. (1994) Ultrastructure of multiple sclerosis. Ultrastruct. Pathol. 18, 3-13
18. Prineas, J. (1975) Pathology of the early lesion in multiple sclerosis. Hum Pathol 6, 531-554
19. Barnett, M. H. and Prineas, J. W. (2004) Relapsing and remitting multiple sclerosis: pathology of the newly forming lesion. Ann. Neurol. 55, 458-468
20. Henderson, A. P. et al. (2009) Multiple sclerosis: distribution of inflammatory cells in newly forming lesions. Ann. Neurol. 66, 739-753
21. Mancardi, G. and Saccardi, R. (2008) Autologous haematopoietic stem-cell transplantation in multiple sclerosis. Lancet Neurol. 7, 626-636
22. Coles, A. J. et al. (1999) Monoclonal antibody treatment exposes three mechanisms underlying the clinical course of multiple sclerosis. Ann. Neurol. 46, 296-304
23. Jones, J. L. and Coles, A. J. (2009) Spotlight on alemtuzumab. Int MS J 16, 77-81
24. Hauser, S. L. et al. (2008) B-cell depletion with rituximab in relapsing-remitting multiple sclerosis. N Engl J Med 358, 676-688
25. Hawker, K. et al. (2009) Rituximab in patients with primary progressive multiple sclerosis: results of a randomized double-blind placebo-controlled multicenter trial. Ann. Neurol. 66, 460-471
26. Filippini, G. et al. (2013) Immunomodulators and immunosuppressants for multiple sclerosis: a network meta-analysis. Cochrane Database Syst Rev 6, CD008933
27. Stys, P. K. et al. (2012) Will the real multiple sclerosis please stand up? Nat. Rev. Neurosci. 13, 507-514
28. Stys, P. K. (2013) Pathoetiology of multiple sclerosis: are we barking up the wrong tree? F1000Prime Rep 5, 20
29. Bremer, J. et al. (2010) Axonal prion protein is required for peripheral myelin maintenance. Nat Neurosci 13, 310-318
30. Collinge, J. et al. (1994) Prion protein is necessary for normal synaptic function. Nature 370, 295-297
31. Kretzschmar, H. A. et al. (1986) Scrapie prion proteins are synthesized in neurons. Am J Pathol 122, 1-5
32. Moya, K. L. et al. (2005) Axonal transport of the cellular prion protein is increased during axon regeneration. J Neurochem 92, 1044-1053
33. Radovanovic, I. et al. (2005) Truncated prion protein and Doppel are myelinotoxic in the absence of oligodendrocytic PrPC. J Neurosci 25, 4879-4888
34. Kretzschmar, H. A. et al. (2000) Function of PrP(C) as a copper-binding protein at the synapse. Arch Virol Suppl 239-249
35. Lau, A. et al. (2015) Octarepeat region flexibility impacts prion function, endoproteolysis and disease manifestation. EMBO Mol Med
36. Khosravani, H. et al. (2008) Prion protein attenuates excitotoxicity by inhibiting NMDA receptors. J. Cell Biol. 181, 551-565
37. Tsutsui, S. et al. (2008) Absence of the cellular prion protein exacerbates and prolongs neuroinflammation in experimental autoimmune encephalomyelitis. Am J Pathol 173, 1029-1041
38. Alfaidy, N. et al. (2013) Prion protein expression and functional importance in developmental angiogenesis: role in oxidative stress and copper homeostasis. Antioxid Redox Signal 18, 400-411
39. Gasperini, L. et al. (2014) Prion Protein and Copper Cooperatively Protect Neurons by Modulating NMDA Receptor Through S-nitrosylation. Antioxid Redox Signal
40. Stys, P. K., You, H. and Zamponi, G. W. (2012) Copper-dependent regulation of NMDA receptors by cellular prion protein: implications for neurodegenerative disorders. J. Physiol. 590, 1357-1368
41. Black, S. A. et al. (2014) Cellular prion protein and NMDA receptor modulation: protecting against excitotoxicity. Front Cell Dev Biol 2, 45
42. You, H. et al. (2012) Aβ damages neurons by altering copper-dependent prion protein regulation of NMDA receptors. Proc. Natl. Acad. Sci. U.S.A. 109, 1737-1742
43. Aguzzi, A., Nuvolone, M. and Zhu, C. (2013) The immunobiology of prion diseases. Nat Rev Immunol
44. Head, M. W. (2013) Human prion diseases: molecular, cellular and population biology. Neuropathology 33, 221-236
45. Kitamoto, T. et al. (1993) A new inherited prion disease (PrP-P105L mutation) showing spastic paraparesis. Ann Neurol 34, 808-813
46. Mead, S. et al. (2013) A novel prion disease associated with diarrhea and autonomic neuropathy. N Engl J Med 369, 1904-1914
47. Aguzzi, A. and O'Connor, T. (2010) Protein aggregation diseases: pathogenicity and therapeutic perspectives. Nat. Rev. Drug Discov. 9, 237-248
48. Aguzzi, A., Baumann, F. and Bremer, J. (2008) The prion's elusive reason for being. Annu Rev Neurosci 31, 439-477
49. Scott, J. R., Davies, D. and Fraser, H. (1992) Scrapie in the central nervous system: neuroanatomical spread of infection and Sinc control of pathogenesis. J Gen Virol 73, 1637-1644
50. Watts, J. C. and Prusiner, S. B. (2014) Mouse models for studying the formation and propagation of prions. J Biol Chem 289, 19841-19849
51. Clarke, A. R., Jackson, G. S. and Collinge, J. (2001) The molecular biology of prion propagation. Philos Trans R Soc Lond B Biol Sci 356, 185-195
52. Jackson, G. S. and Clarke, A. R. (2000) Mammalian prion proteins. Curr Opin Struct Biol 10, 69-74
53. Surewicz, W. K. and Apostol, M. I. (2011) Prion protein and its conformational conversion: a structural perspective. Top Curr Chem 305, 135-167
54. Narang, H. K. and Field, E. J. (1973) An electron-microscopic study of multiple sclerosis biopsy material: some unusual inclusions. J Neurol Sci 18, 287-300
55. Narang, H. K. and Field, E. J. (1973) Paramyxovirus like tubules in multiple sclerosis biopsy material. Acta Neuropathol 25, 281-290
56. DeArmond, S. J. et al. (1985) Identification of prion amyloid filaments in scrapie infected brain. Cell 41, 221-235
57. Nennesmo, I. et al. (1997) Multiple sclerosis and amyloid deposits in the white matter of the brain. Acta Neuropathol 93, 205-209
58. Field, E. J. (1969) Slow virus infections of the nervous system. Int Rev Exp Pathol 8, 129-239
59. Wojtowicz, S. (1993) Multiple sclerosis and prions. Med Hypotheses 40, 48-54
60. Kurtzke, J. F. (2005) Epidemiology and etiology of multiple sclerosis. Phys Med Rehabil Clin N Am 16, 327-349
61. Kurtzke, J. F. and Hyllested, K. (1979) Multiple sclerosis in the Faroe Islands: I. Clinical and epidemiological features. Ann Neurol 5, 6-21
62. Hawkes, C. H. (2002) Is multiple sclerosis a sexually transmitted infection? J Neurol Neurosurg Psychiatry 73, 439-443
63. Hawkes, C. H. et al. (2012) MRI and visual-evoked potentials in partners of multiple sclerosis patients. Acta Neurol Scand 125, 424-430
64. Diack, A. B. et al. (2014) Variably protease-sensitive prionopathy, a unique prion variant with inefficient transmission properties. Emerg Infect Dis 20, 1969-1979
65. Notari, S. et al. (2014) Transmission characteristics of variably protease-sensitive prionopathy. Emerg Infect Dis 20, 2006-2014
66. Gambetti, P. et al. (2008) A novel human disease with abnormal prion protein sensitive to protease. Ann Neurol 63, 697-708
67. Jansen, C. et al. (2010) The first case of protease-sensitive prionopathy (PSPr) in The Netherlands: a patient with an unusual GSS-like clinical phenotype. J Neurol Neurosurg Psychiatry 81, 1052-1055
68. Beringue, V. et al. (2008) Prominent and persistent extraneural infection in human PrP transgenic mice infected with variant CJD. PLoS One 3, e1419
69. Bishop, M. T. et al. (2006) Predicting susceptibility and incubation time of human-to-human transmission of vCJD. Lancet Neurol 5, 393-398
70. Trapp, B. D. and Stys, P. K. (2009) Virtual hypoxia and chronic necrosis of demyelinated axons in multiple sclerosis. Lancet Neurol. 8, 280-291
71. Coombs, B. D. et al. (2004) Multiple sclerosis pathology in the normal and abnormal appearing white matter of the corpus callosum by diffusion tensor imaging. Mult Scler 10, 392-397
72. Peden, A. H. et al. (2014) The prion protein protease sensitivity, stability and seeding activity in variably protease sensitive prionopathy brain tissue suggests molecular overlaps with sporadic Creutzfeldt-Jakob disease. Acta Neuropathol Commun 2, 152
73. Safar, J. et al. (1998) Eight prion strains have PrP(Sc) molecules with different conformations. Nat Med 4, 1157-1165
74. White, A. R. et al. (2003) Monoclonal antibodies inhibit prion replication and delay the development of prion disease. Nature 422, 80-83
75. Atarashi, R. et al. (2011) Ultrasensitive human prion detection in cerebrospinal fluid by real-time quaking-induced conversion. Nat Med 17, 175-178
76. Orru, C. D. et al. (2015) Bank Vole Prion Protein As an Apparently Universal Substrate for RT-QuIC-Based Detection and Discrimination of Prion Strains. PLoS Pathog 11, e1004983
77. Gambetti, P., Puoti, G. and Zou, W. Q. (2011) Variably protease-sensitive prionopathy: a novel disease of the prion protein. J Mol Neurosci 45, 422-424
78. Prusiner, S. B. (1984) Prions: novel infectious pathogens. Adv Virus Res 29, 1-56
79. Prusiner, S. B. (1993) Prion encephalopathies of animals and humans. Dev Biol Stand 80, 31-44
80. Prusiner, S. B. (1998) The prion diseases. Brain Pathol 8, 499-513
81. Zlotnik, I. and Rennie, J. C. (1965) Experimental transmission of mouse passaged scrapie to goats, sheep, rats and hamsters. J Comp Pathol 75, 147-157
82. Fischer, M. et al. (1996) Prion protein (PrP) with amino-proximal deletions restoring susceptibility of PrP knockout mice to scrapie. EMBO J 15, 1255-1264
83. Bueler, H. et al. (1992) Normal development and behaviour of mice lacking the neuronal cell-surface PrP protein. Nature 356, 577-582
84. Silveira, J. R. et al. (2005) The most infectious prion protein particles. Nature 437, 257-261
85. Beekes, M. and McBride, P. A. (2007) The spread of prions through the body in naturally acquired transmissible spongiform encephalopathies. FEBS J 274, 588-605
86. McBride, P. A. et al. (2001) Early spread of scrapie from the gastrointestinal tract to the central nervous system involves autonomic fibers of the splanchnic and vagus nerves. J Virol 75, 9320-9327
87. Weissmann, C. and Flechsig, E. (2003) PrP knock-out and PrP transgenic mice in prion research. Br Med Bull 66, 43-60
88. Bruce, M. E. (1993) Scrapie strain variation and mutation. Br Med Bull 49, 822-838
89. Korth, C. et al. (2003) Abbreviated incubation times for human prions in mice expressing a chimeric mouse-human prion protein transgene. Proc Natl Acad Sci USA 100, 4784-4789
90. Telling, G. C. (2011) Transgenic mouse models and prion strains. Top Curr Chem 305, 79-99
91. Shi, Q. et al. (2015) Successive passaging of the scrapie strains, ME7-ha and 139A-ha, generated by the interspecies transmission of mouse-adapted strains into hamsters markedly shortens the incubation times, but maintains their molecular and pathological properties. Int J Mol Med
92. D'Castro, L. et al. (2010) Isolation of proteinase K-sensitive prions using pronase E and phosphotungstic acid. PLoS One 5, e15679
93. Cannon, A. et al. (2014) Concurrent variably protease-sensitive prionopathy and amyotrophic lateral sclerosis. Acta Neuropathol 128, 313-315
94. Kimberlin, R. H. and Walker, C. (1977) Characteristics of a short incubation model of scrapie in the golden hamster. J Gen Virol 34, 295-304
95. Cronier, S. et al. (2008) Detection and characterization of proteinase K-sensitive disease-related prion protein with thermolysin. Biochem J 416, 297-305
96. Kovacs, G. G. et al. (2013) Rapidly progressive dementia with thalamic degeneration and peculiar cortical prion protein immunoreactivity, but absence of proteinase K resistant PrP: a new disease entity? Acta Neuropathol Commun 1, 72
97. Collins, S. J. et al. (2005) Extended period of asymptomatic prion disease after low dose inoculation: assessment of detection methods and implications for infection control. Neurobiol Dis 20, 336-346
98. Wadsworth, J. D. et al. (2001) Tissue distribution of protease resistant prion protein in variant Creutzfeldt-Jakob disease using a highly sensitive immunoblotting assay. Lancet 358, 171-180
99. McLennan, N. F. et al. (2004) Prion protein accumulation and neuroprotection in hypoxic brain damage. Am J Pathol 165, 227-235
100. Weise, J. et al. (2004) Upregulation of cellular prion protein (PrPc) after focal cerebral ischemia and influence of lesion severity. Neurosci Lett 372, 146-150
101. Itoyama, Y., Sternberger, N. H. and de F. Webster, H., et. al. (1980) Immunocytochemical observations on the distribution of myelin-associated glycoprotein and myelin basic protein in multiple sclerosis lesions. Ann Neurol 7, 167-177
102. Calabrese, M. et al. (2015) Exploring the origins of grey matter damage in multiple sclerosis. Nat Rev Neurosci 16, 147-158
103. Wegner, C. et al. (2006) Neocortical neuronal, synaptic, and glial loss in multiple sclerosis. Neurology 67, 960-967
104. Haeberle, A. M. et al. (2000) Synaptic prion protein immuno-reactivity in the rodent cerebellum. Microsc Res Tech 50, 66-75
105. Geurts, J. J. et al. (2009) Gray matter pathology in (chronic) MS: Modern views on an early observation. J. Neurol. Sci. 282, 12-20
106. DeArmond, S. J. et al. (1987) Changes in the localization of brain prion proteins during scrapie infection. Neurology 37, 1271-1280
107. Haritani, M., Spencer, Y. I. and Wells, G. A. (1994) Hydrated autoclave pretreatment enhancement of prion protein immunoreactivity in formalin-fixed bovine spongiform encephalopathy-affected brain. Acta Neuropathol 87, 86-90
108. van Keulen, L. J. et al. (1995) Immunohistochemical detection and localization of prion protein in brain tissue of sheep with natural scrapie. Vet Pathol 32, 299-308
109. Kascsak, R. J. et al. (1987) Mouse polyclonal and monoclonal antibody to scrapie associated fibril proteins. J Virol 61, 3688-3693
110. Zou, W. Q. and Cashman, N. R. (2002) Acidic pH and detergents enhance in vitro conversion of human brain PrPC to a PrPSc-like form. J Biol Chem 277, 43942-43947
111. Xiao, X., Yuan, J. and Zou, W. Q. (2012) Isolation of soluble and insoluble PrP oligomers in the normal human brain. J Vis Exp
112. Falsig, J. et al. (2012) Prion pathogenesis is faithfully reproduced in cerebellar organotypic slice cultures. PLoS Pathog 8, e1002985
113. Campeau, J. L. et al. (2013) Early increase and late decrease of purkinje cell dendritic spine density in prion-infected organotypic mouse cerebellar cultures. PLoS One 8, e81776
114. Falsig, J. and Aguzzi, A. (2008) The prion organotypic slice culture assay-POSCA. Nat Protoc 3, 555-562
115. Micu, I. et al. (2006) NMDA receptors mediate Ca accumulation in central nervous system myelin during chemical ischemia. Nature 439, 988-992
116. Cramm, M. et al. (2015) Characteristic CSF prion seeding efficiency in humans with prion diseases. Mol Neurobiol 51, 396-405
117. Goni, F. et al. (2015) Mucosal immunization with an attenuated Salmonella vaccine partially protects white-tailed deer from chronic wasting disease. Vaccine 33, 726-733
118. Trojano, M. and Paolicelli, D. (2001) The differential diagnosis of multiple sclerosis: classification and clinical features of relapsing and progressive neurological syndromes. Neurol Sci 22 Suppl 2, S98-102
119. Kidd D, Barker G J, Tofts P S, Gass A, Thompson A J, McDonald W I, and Miller D H. (1997) The transverse magnetisation decay characteristics of longstanding lesions and normal-appearing white matter in multiple sclerosis. J Neurol. Feb; 244(2):125-30
120. Micu I, Plemel J R, Caprariello A V, Nave K A and Stys P K. (2018) Axo-myelinic neurotransmission: a novel mode of cell signalling in the central nervous system. Nat Rev Neurosci, Jan; 19(1):49-58
121. Korth et al. Acridine and phenothiazine derivatives as pharmacotherapeutics for prion disease. Proc Natl Acad Sci USA. 2001 Aug. 14; 98(17):9836-41
120. Doh-ura et al. Treatment of transmissible spongiform encephalopathy by intraventricular drug infusion in animal models. J Virol. 2004 May; 78(10):4999-5006
121. De Luigi et al. The efficacy of tetracyclines in peripheral and intracerebral prion infection. PLoS One. 2008 Mar. 26; 3(3):e1888
122. Karapetyan et al. Unique drug screening approach for prion diseases identifies tacrolimus and astemizole as antiprion agents. Proc Natl Acad Sci USA. 2013 Apr. 23; 110(17):7044-9
123. Herrmann et al. Structure-based drug design identifies polythiophenes as antiprion compounds. Sci Transl Med. 2015 Aug. 5; 7(299):299ra123
124. Wagner et al. Anle138b: a novel oligomer modulator for disease-modifying therapy of neurodegenerative diseases such as prion and Parkinson's disease. Acta Neuropathol. 2013 June; 125(6):795-813

1. A method of identifying a subject having, or at risk of developing, multiple sclerosis (MS), the method comprising:
detecting the presence or absence of an abnormal prion protein conformer (PrP^{MS}) in a sample from the subject, wherein the presence of the abnormal prion protein conformer in the sample is indicative of the subject having MS or an increased risk of developing MS, wherein PrP^{MS} is a transmissible, proteinase K-sensitive conformer of PrP.

2. The method of claim 1, wherein the abnormal prion protein conformer is absent in a control sample from a subject without MS.

3. The method of claim 1, wherein the abnormal prion protein conformer forms deposits of β-sheet rich amyloid.

4. The method of claim 1, wherein the abnormal prion protein conformer causes transmission of MS pathology to a test subject when injected intracerebrally into the test subject.

5. The method of claim 4, wherein the abnormal prion protein conformer causes the test subject to develop brain atrophy, amyloid plaques, demyelination, abnormal behavior and/or ventriculomegaly.

6. The method of claim 4, wherein the test subject is a transgenic mouse expressing or overexpressing prion protein (PrP), optionally human prion protein.

7. The method of claim 1, wherein pronase E digestion of PrP^{MS} generates smaller molecular weight fragments of prion protein that are resistant to further digestion and wherein PrP^{MS} is more resistant to pronase E digestion relative to prion protein from a control sample from a subject without MS.

8. The method of claim 1, wherein the sample is a tissue sample, a cerebrospinal fluid (CSF) sample, or a blood sample.

9.-10. (canceled)

11. The method of claim 8, wherein the tissue sample is a brain tissue sample and the method comprises detecting the abnormal prion protein conformer in the brain tissue sample in extracellular space, around vessels, oligodendrocytes, oligodendrocyte precursor cells, microglia, astrocytes, neurons, cortex and/or white matter.

12.-13. (canceled)

14. The method of claim 1, wherein detecting the presence or absence of the abnormal prion protein conformer in the sample comprises spectral analysis.

15.-17. (canceled)

18. The method of claim 1, wherein detecting the presence or absence of the abnormal prion protein conformer in the sample comprises RT-QuIC, immunohistochemistry or Photo-Induced Cross-Linking of Unmodified Proteins (PICUP) and/or increasing the number of abnormal prion protein conformers in the sample using protein-misfolding cyclic amplification (PMCA).

19.-39. (canceled)

40. A non-human animal model of multiple sclerosis (MS), produced by administering abnormal prion protein conformer (PrP^{MS}) to a non-human animal, wherein PrP^{MS} is a transmissible, proteinase K-sensitive conformer of PrP.

41. (canceled)

42. The non-human animal model of claim 40, wherein the non-human animal is a non-human primate or a rodent.

43. The non-human animal model of claim 40, wherein the non-human animal develops one or more signs or symptoms of MS after administration of the abnormal prion protein conformer, wherein the signs or symptoms of multiple sclerosis are selected from the presence of amyloid plaques, brain atrophy, demyelination, axonal damage, ventriculomegaly and motor/pain perception/cognitive/visual abnormalities.

44. (canceled)

45. The non-human animal model of claim 40, produced by administering abnormal prion protein conformer (PrP^{MS}) to the non-human organism by intracerebral or intraperitoneal injection.

46. The non-human animal model of claim 40, produced by administering to the non-human organism a brain homogenate, spinal chord homogenate, CSF or other biological sample comprising abnormal prior protein conformer (PrP^{MS}), optionally wherein the brain homogenate, spinal chord homogenate, CSF or other biological sample is from a subject with MS.

47. The non-human animal model of claim 40, wherein the non-human animal is a transgenic animal that overexpresses prion protein.

48.-52. (canceled)

53. A method of identifying candidate compounds for the detection or treatment of multiple sclerosis (MS), the method comprising:
(a) administering a test compound to the non-human animal model of claim 40; and
(b) detecting a biological effect of the test compound on the non-human animal model.

54. (canceled)

55. The method of claim 53, wherein the biological effect is a reduction in the presence of abnormal prion protein conformer and/or amyloid-like deposits in the non-human model organism relative to a control, or an increase or decrease in one or more signs or symptoms of MS, wherein the signs or symptoms of MS are selected from the presence of amyloid plaques, demyelination, brain atrophy, ventriculomegaly and abnormal animal behavior.

56.-73. (canceled)

74. A method of producing a non-human animal model of multiple sclerosis (MS), the method comprising administering abnormal prion protein conformer (PrP^{MS}) to the non-human animal, wherein PrP^{MS} is a transmissible, proteinase K-sensitive conformer of PrP, wherein the non-human animal develops one or more signs or symptoms of MS after administration of PrP^{MS}.
